(19) United States
(12) Patent Application Publication
(10) Pub. No.: US 2023/0158068 A1
(43) Pub. Date: May 25, 2023

(54) METHOD AND APPARATUS FOR THREE DIMENSIONAL ALVEOLAR LUNG MODEL

(71) Applicant: University of Hertfordshire Higher Education Corporation, Hatfield (GB)

(72) Inventors: Victoria Hutter, Hatfild (GB); Abigail Martin, Hatfield (GB); Ewelina Hoffman, Hatfield (GB)

(21) Appl. No.: 17/995,736

(22) PCT Filed: Apr. 6, 2021
(86) PCT No.: PCT/GB2021/050841

§ 371 Date: Oct. 7, 2022

(30) Foreign Application Priority Data
Apr. 7, 2020 (GB) 2005154.6

(51) Int. Cl. A61K 35/12

(52) U.S. Cl. CPC A61K 35/12; A61K 35/42

(57) ABSTRACT

The invention relates to a human in vitro model and a method of constructing the same to mimic the alveolar region of the airways to assess the respiratory toxicology and/or physiological and/or biological response of inhaled products, chemicals and particles. There is provided a three-dimensional in vitro alveolar lung model and a method of constructing the same comprising a culture well provided with a membrane configured to separate the culture well into a first compartment and a second compartment, wherein the membrane has first side configured form a wall of the first compartment and a second side configured to form a wall of the second compartment, wherein alveolar type I epithelial cells are provided in the first compartment and alveolar macrophage-like cells are provided in the second compartment.

### FIELD OF THE INVENTION

The present invention relates generally to the fields of microbiology, pulmonology, respiratory physiology, infectious disease, immunology, cell biology, toxicology, cancer, environmental microbiology, bioengineering, biotechnology, vaccine development, adjuvant development, therapeutic development, and drug development. The present invention particularity relates to a human in vitro model and a method of constructing the same to mimic the alveolar region of the airways to assess the respiratory response of inhaled products and those administered by other delivery routes which result in the products being present in the systemic circulation.

### BACKGROUND OF THE INVENTION

Human lungs are constantly exposed to environmental and chemical substances in the air. Certain inhaled substances or particles can cause damage to the lung (e.g. asbestos) or be taken up into the blood stream and cause toxicity in the body. New chemicals and medicines that could enter the lungs must be tested for safety before they can be marketed. Aerosolised substances with a particle size below around 1 μm to 2 μm will deposit in the deep lung (alveolar region) where gas exchange occurs. It is now established that immunological responses in the alveolar region are key to understanding the consequence of exposure (adaptive or adverse) and hence to predicting the safety of an inhaled compound.

Currently, inhaled safety assessment and assessment of biological responses to inhaled aerosols often involves animal studies (rats, mice, dogs). However, these models are often costly, time consuming and do not provide a good representation of human lungs, leading to inaccurate assessment of safety. Additionally, the mechanistic understanding of the cellular effects involved is still limited. There is a drive to move towards non-animal methods for toxicology assessment and other respiratory responses but currently there is no regulatory standard for in vitro inhaled safety assessment.

Whilst, human airway in vitro cell culture models are widely used for assessing the toxicity of inhaled compounds, these models generally only involve one type of cell (epithelial) and are not representative of the complex nature of the lung. The majority of human airway epithelial in vitro cell culture models available represent the upper (conducting airways) and not the physiology of the alveolar epithelium where exposure for inhaled medicines/chemicals occurs.

A publication by Klein et al., in 2013 proposed a tetra culture model composed of an alveolar type II epithelial cell line (A549), differentiated macrophage-like cells (THP1), mast cells (HMC-1) and endothelial cells (EA.hy 926), which made it possible that the model could then be exposed at the air-liquid interface. However, it was observed that the cells formed heterogeneous colonies under submerged conditions: this leads to overestimation of observed effects in the results for instance for ROS (reactive oxygen species) production and IL-8 secretion. Furthermore, Klein's model cannot be used for the evaluation of sensitizing effects due to the lack of relevant competent cells and does not allow for cell migration through the membrane, due to the reduced pores size. WO2018/122219 (LUXEMBOURG INSTITUTE OF SCIENCE AND TECHNOLOGY) describes a similar tetra-culture system. However, in both of these models the alveolar type II epithelial cells (A549) selected are not able to form tight junctions and hence the model cannot be used to study the permeation of substances.

A publication by Kletting et al., in 2019 (https://www.altex.org/index.php/altex/article/view/89/842) describes the co-culture of hAELVi (alveolar type I epithelial cells) with the THP-1 cell lines (monocyte derived macrophages) for use in safety and permeability assessment. However, the THP-1 cell line selected represents blood-derived monocytes and is not representative of the alveolar macrophage lineage found in the alveolar airspace. Furthermore, the two cell types were combined in a single culture compartment making analysis of each distinct cell population response difficult and limits the functionality and usability of the model.

US2013344501A1 (CRABBE AURELIE; NICKERSON CHERYL ANNE; SARKER SHAMEEMA) describes methods of producing a three-dimensional, physiologically relevant immune tissue system. This methodology uses a bioreactor to culture A549 and U937 cells on porous microcarrier beads in a low shear environment. This creates 3D-spheres of A549 and U937 cells which represent some functionality of the environment of the alveolus. However, A549 cells are an alveolar type II epithelial cell line and constitute approximately 5% of the area of the alveolar epithelium, and hence do not comprise the main cell type in the epithelial barrier to permeation of drugs/chemicals/particles found in vivo. Furthermore, in the model the two cell types are combined on a single scaffold and are not able to be separated once constructed. Similar to the model described by Kletting, analysis of each distinct cell population response is difficult, and this limits the functional understanding and practical application of the model.

All previous airway epithelial-immune models so far proposed for respiratory safety assessment and assessment of biological responses to inhaled aerosols have been formed using either bronchial epithelial cell lines, alveolar type II cells (which do not form tight junctions) and/or monocyte derived macrophages originating from blood and which do not represent the alveolar macrophage and are less relevant in the context of small airway responses. Therefore, there is a need for a relevant tool to study small airway responses and for predicting the topical safety of inhaled products as well as the systemic safety of products administered via other delivery routes on the human lung.

### SUMMARY OF THE INVENTION

According to a first aspect of the present invention there is provided a method for preparing a three-dimensional in vitro alveolar lung model comprising a culture well provided with a membrane configured to separate the culture well into a first compartment and a second compartment, wherein the membrane has a first side configured form a wall of the first compartment and a second side configured to form a wall of the second compartment, wherein alveolar type I epithelial cells are provided in the first compartment and alveolar macrophage-like cells are provided in the second compartment.

Preferably the first compartment is configured to be exposed to an air-liquid interface and the second compartment configured to be submerged in a culture medium. In an alternative the second compartment is configured to be exposed to an air-liquid interface and the first compartment configured to be submerged in a culture medium. In a further alternative both the first and second compartments are configured to be submerged in a culture medium.

Preferably the first compartment comprises an apical compartment and the second compartment comprises a basolateral compartment,

Preferably the first side of the membrane is an apical side and the second side of the membrane is a basolateral side.

Preferably the alveolar type I epithelial cells are hAELVi cells.

In a further alternative a combination of both alveolar type I epithelial cells and alveolar type II epithelial cells are provided in the first compartment, preferably a combination of hAELVi cells and A549 cells.

Preferably the method comprises preparing a co-culture of a) alveolar type I epithelial cells or combination of alveolar type I and type II epithelial cells and b) alveolar macrophage-like cells.

Preferably the method of preparing the co-culture comprises the following step sequence:
i) seeding the first side of the membrane with the alveolar type I epithelial cells or combination of alveolar type I and type II epithelial cells;
ii) introducing the membrane into a first culture well such that the type I epithelial cells or combination of alveolar type I and type II epithelial cells are present in the first compartment, preferably at the air-liquid interface (ALI);
iii) introducing a first culture medium into a first culture well;
iv) culturing the alveolar type I epithelial cells or combination of alveolar type I and type II epithelial cells;
v) seeding a second culture well with leukocyte cells in a second culture medium;
vi) differentiating the leukocyte cells to alveolar macrophage-like cells; and
vii) removing the membrane with the cultured alveolar type I epithelial cells or combination of alveolar type I and type II epithelial cells from the first culture well and introducing the membrane with the cultured alveolar type I epithelial cells or combination of alveolar type I and type II epithelial cells into the second culture medium of the second culture well such that the alveolar macrophage-like cells, present in the second compartment are preferably submerged in the second culture medium, and the alveolar type I epithelial cells or combination of alveolar type I and type II epithelial cells are present in the first compartment, preferably at the ALI.

In an alternative the method of preparing the co-culture comprises the following step sequence:
i) seeding the first side of the membrane with the alveolar type I epithelial cells or combination of alveolar type I and type II epithelial cells;
ii) seeding the second side of the membrane with leukocyte cells;
iii) introducing a second culture medium into the culture well;
iv) introducing the membrane into a culture well such that the alveolar type I epithelial cells or combination of alveolar type I and type II epithelial cells are present in the first compartment, preferably at the air-liquid interface (ALI);
v) introducing a first culture medium into the culture well;
vi) culturing the alveolar type I epithelial cells or combination of alveolar type I and type II epithelial cells; and
vii) differentiating the leukocyte cells to alveolar macrophage-like cells.

Seeding is defined as introducing a defined amount (volume or cell number) of a cell suspension into a container (such as the culture cell) or onto a surface (such as the membrane).

Preferably the first side of the membrane is seeded with between 1×10⁴ and 5×10⁵ alveolar type I epithelial cells or combination of alveolar type I and type II epithelial cells/cm², more preferably 1×10⁵ alveolar type I epithelial cells or combination of alveolar type I and type II epithelial cells/cm².

Preferably the first side of the membrane, which is seeded with the alveolar type I epithelial cells or combination of alveolar type I and type II epithelial cells, is raised to the air-liquid interface after seeding.

Preferably the alveolar type I epithelial cells or combination of alveolar type I and type II epithelial cells are cultured at the air liquid interface.

Preferably the culture well or second side or basolateral side of the membrane is seeded with 1.75×10⁵ leukocyte cells/cm².

Preferably the alveolar type I epithelial cells or combination of alveolar type I and type II epithelial cells are cultured for between 4-28 days, preferably for 10 days.

Culturing is defined as the maintenance or growth of cells in controlled conditions outside of their native environment.

Preferably the method further comprises differentiating the leukocyte cells to alveolar macrophage-like cells.

Differentiating is defined as the process through which a cell undergoes changes in gene expression to become a more specific type of cell.

Preferably the leukocyte cells are differentiated to alveolar macrophage-like cells with phorbol-12-myristate-13-acetate (PMA) or with 1, 25 dihydroxyvitamin D3, most preferably differentiated with PMA.

Preferably the first culture medium comprises Dulbecco's Modified Eagle's Medium (DMEM), Dulbecco's Modified Eagle's Medium/Ham's F12 (DMEM/F12) (50:50), Roswell Park Memorial Institute-1640 (RPMI), Small Airways Growth Medium (SAGM) (Lonza), human airway epithelial cell medium (hAEC), MucilAir culture medium, SmallAir culture medium (Epithelix) or human alveolar epithelium cell culture medium (huAEC) (InSCREENeX) and more preferably RPMI or huAEC.

Preferably the first culture medium comprises huAEC medium (InSCREENeX), huAEC basal supplements (bovine pituitary extract, insulin, gentamicin sulfate and amphotericin (GA-1000), retinoic acid, bovine serum albumin-fatty acid free (BSA-FAF), transferrin, triiodo-L-thyronine (T3), epinephrine, recombinant human epidermal growth factor (rhEGF)),InSCREENeX), FBS and an antibiotic/antimitotic agent.

Preferably the antibiotic/antimitotic agent is selected from one or more of penicillin, streptomycin, gentamicin and amphotericin.

Preferably the second culture medium comprises DMEM, DMEM/F12 (50:50), RPMI, SAGM (Lonza), hAEC, MucilAir, SmallAir (Epithelix) or huAEC (InSCREENeX) and more preferably RPMI or huAEC.

Preferably the second culture medium comprises RPMI, FBS, L-glutamine and an antibiotic/antimitotic agent.

Preferably the antibiotic/antimitotic agent is selected from one or more of penicillin, streptomycin, gentamicin and amphotericin.

Preferably the membrane comprises a porous membrane.

Preferably the porous membrane is configured for potential migration of the alveolar macrophage-like cells between the second and first compartments, preferably between the basolateral compartment and the apical compartment.

Preferably the porous membrane is provided with a plurality of pores, preferably the pores are between about 0.4-10 μm in diameter, more preferably between about 0.4-8 μm in diameter, and even more preferably between about 0.4-3 μm in diameter.

Optionally, a perfusion system is provided to allow for circulation of the first and/or second culture mediums, in one alternative the perfusion system is an external perfusion system.

Preferably the membrane is pre-treated for optimal cell growth.

Preferably the pre-treatment comprises a coating or coating methodology.

Preferably the coating is provided on the first side of the membrane, preferably the coating is provided on the apical side of the membrane, preferably the coating is provided on the growth surface of the alveolar type I epithelial cells or combination of alveolar type I and type II epithelial cells.

Preferably the coating comprises a biological and/or synthetic polymer.

Preferably the coating comprises collagen, gelatin, laminin fibronectin, poly-L-lysine or serum.

Preferably the coating is selected from collagen, gelatin, laminin fibronectin, poly-L-lysine or serum.

Preferably the coating is configured to optimise cell attachment, proliferation and function for the alveolar type I cells or combination of alveolar type I and type II epithelial cells to exhibit morphology and functionality that most closely resembles that of alveolar type I cells or combination of alveolar type I and type II epithelial cells in their native environment. Preferably the leukocyte cells are monocytes.

Preferably the leukocyte cells are lung derived monocytes.

Preferably the leukocyte cells are U937 cells.

Preferably the alveolar macrophage-like cells are U937 cells differentiated with PMA (phorbol-12-myristate-13-acetate) or with 1, 25 dihydroxyvitamin D3, most preferably differentiated with PMA.

Preferably the differentiation is performed over several days: preferably 1-7 days and more preferably 3 days.

Preferably step vii) takes place about 7-14 days after the seeding of the alveolar type I epithelial cells or combination of alveolar type I and type II epithelial cells and after about 24 hours of differentiation of the alveolar macrophage-like cells.

Preferably all cells are immortalised mammalian cell lines, which are cells more phenotypically and functionally stable than primary cells and are more preferably immortalised human cell lines.

Preferably the alveolar type I epithelial cells are configured to form tight junctions and a polarised cell layer. This represents as close as is possible the barrier present to transport of inhaled chemicals/particles in the small airways/alveolus. This property is essential to be able to determine the systemic toxicity, biological response, therapeutic response, pharmacological response and potential absorption of molecules from the lungs into the body for the best prediction of toxicity/therapeutic effectiveness.

Preferably the alveolar macrophage-like cells are configured to participate in defence mechanisms by ingesting foreign materials by phagocytosis. This represents the functionality of the cells in vivo. This property indicates their ability to respond to chemical/particulate stimuli and the downstream signalling associated with the response. This functionality provides the best prediction of inflammatory responses and associated toxicity.

Preferably the alveolar type I epithelial cells are hAELVi alveolar type I epithelial cells and the alveolar macrophage-like cells are U937 cells, differentiated with PMA. This combination represents the two essential cell types present in the alveoli which provide the first responses to inhaled chemicals/particulates. Furthermore, it is established that alveolar macrophages and alveolar epithelial cell cross-talk is one of the key determinants in cascading inflammatory responses in the airways. Modelling the interaction between the cell types involved in the primary response to an inhaled compound provides a platform to determine the downstream response pathways and determine whether an adaptive/adverse response to an inhaled stimulus would be initiated.

Compared with the co-culture model of Kletting et al., the present model utilises U937 cells instead of THP-1 cells for the alveolar macrophage-like component. There are no human alveolar macrophage-like cell lines currently in existence, however the U937 cell line isolated from a human pleural effusion is a monocytic cell line originating from the lung with the capacity to most closely resemble the alveolar macrophage rather than the THP-1 cells which are from a blood monocyte population. Lung-derived macrophages are from a different lineage to blood monocyte-derived macrophages and hence they possess different characteristics and functionalities. Therefore, the use of U937 cells in the present model more precisely mimics the in vivo situation and provides the closest representation of an alveolar macrophage-like cells from a co-culture cell line model.

Compared with other U937 co-culture models, the present invention uses alveolar type I epithelial cells or a combination of both alveolar type I and type II epithelial cells rather than alveolar type II cells (e.g. A549) on their own. Alveolar type I epithelial cells comprise approximately 90% of the epithelial cell surface of the alveolus. Therefore, the present model mimics more precisely the in vivo situation where alveolar type I epithelial cells form a tight monolayer of cells in the alveolus and constitute the primary cell barrier to permeation of substances between the airspace and blood supply in the alveolus.

In one alternative the porous membrane separating the first or apical and second or basolateral compartments is a Transwell® or Snapwell® insert. Advantageously, the cell types are provided in different compartments (allowing the diffusion of chemical mediators between the cells and with the potential for migration through the porous membrane to more precisely mimic the in vitro conditions) making analysis of the responses of each cell population easier to assess and attribute more specific functional determination of response. For example, the alveolar type I epithelial cells or the combination of both alveolar type I and type II epithelial cells can respond to biochemical signals released by the alveolar macrophage-like cells and vice versa.

According to a second aspect of the invention there is provided a three-dimensional in vitro alveolar airway model constructed according to the method of the first aspect of the present invention.

According to a third aspect of the present invention there is provided a three-dimensional in vitro alveolar lung model comprising a culture well provided with a membrane configured to separate the culture well into a first compartment and a second compartment, wherein the membrane has a first side configured form a wall of the first compartment and a second side configured to form a wall of the second compartment, wherein alveolar type I epithelial cells are provided in the first compartment and alveolar macrophage-like cells are provided in the second compartment.

Preferably the first compartment is configured to be exposed to an air-liquid interface and the second compartment configured to be submerged in a culture medium. In an alternative the second compartment is configured to be exposed to an air-liquid interface and the first compartment configured to be submerged in a culture medium. In a further alternative both the first and second compartments are configured to be submerged in a culture medium.

Preferably the first compartment comprises an apical compartment and the second compartment comprises a basolateral compartment.

Preferably the first side of the membrane is an apical side and the second side of the membrane is a basolateral side.

Preferably the alveolar type I epithelial cells are hAELVi cells.

In a further alternative a combination of both alveolar type I epithelial cells and alveolar type II epithelial cells are provided in the first compartment, preferably a combination of hAELVi cells and A549 cells.

Preferably the alveolar macrophage-like cells comprise differentiated leukocyte cells.

Preferably the alveolar macrophage-like cells comprise leukocyte cells differentiated with phorbol-12-myristate-13-acetate (PMA) or with 1, 25 dihydroxyvitamin D3, most preferably differentiated with PMA.

Preferably the culture medium comprises Dulbecco's Modified Eagle's Medium (DMEM), Dulbecco's Modified Eagle's Medium/Ham's F12 (DMEM/F12) (50:50), Roswell Park Memorial Institute-1640 (RPMI), Small Airways Growth Medium (SAGM) (Lonza), human airway epithelial cell medium (hAEC), MucilAir culture medium, SmallAir culture medium (Epithelix) or human alveolar epithelium cell culture medium (huAEC) (InSCREENeX) and more preferably RPMI or huAEC.

Preferably the culture medium comprises huAEC medium (InSCREENeX), huAEC basal supplements (bovine pituitary extract, insulin, gentamicin sulfate and amphotericin (GA-1000), retinoic acid, bovine serum albumin-fatty acid free (BSA-FAF), transferrin, triiodo-L-thyronine (T3), epinephrine, recombinant human epidermal growth factor (rhEGF)),InSCREENeX), FBS and an antibiotic/antimitotic agent.

Preferably the antibiotic/antimitotic agent is selected from one or more of penicillin, streptomycin, gentamicin and amphotericin.

Alternatively, the culture medium comprises DMEM, DMEM/F12 (50:50), RPMI, SAGM (Lonza), hAEC, MucilAir, SmallAir (Epithelix) or huAEC (InSCREENeX) and more preferably RPMI or huAEC.

Preferably the culture medium comprises RPMI, FBS, L-glutamine and an antibiotic/antimitotic agent.

Preferably the antibiotic/antimitotic agent is selected from one or more of penicillin, streptomycin, gentamicin and amphotericin.

Preferably the membrane comprises a porous membrane.

Preferably the porous membrane is configured for potential migration of the alveolar macrophage-like cells between the second and first compartments, preferably between the basolateral compartment and the apical compartment.

Preferably the porous membrane is provided with a plurality of pores, preferably the pores are between about 0.4-10 μm in diameter, more preferably between about 0.4-8 μm in diameter, and even more preferably between about 0.4-3 μm in diameter.

Optionally, a perfusion system is provided to allow for circulation of the first and/or second culture mediums, in one alternative the perfusion system is an external perfusion system.

Preferably the membrane is pre-treated for optimal cell growth.

Preferably the pre-treatment comprises a coating or coating methodology.

Preferably the coating is provided on the first side of the membrane, preferably the coating is provided on the apical side of the membrane, preferably the coating is provided on the growth surface of the alveolar type I epithelial cells or combination of alveolar type I and type II epithelial cells.

Preferably the coating comprises a biological and/or synthetic polymer.

Preferably the coating comprises collagen, gelatin, laminin fibronectin, poly-L-lysine or serum.

Preferably the coating is selected from collagen, gelatin, laminin fibronectin, poly-L-lysine or serum.

Preferably the coating is configured to optimise cell attachment, proliferation and function for the alveolar type I cells or combination of alveolar type I and type II epithelial cells to exhibit morphology and functionality that most closely resembles that of alveolar type I cells or combination of alveolar type I and type II epithelial cells in their native environment.

Preferably the leukocyte cells are monocytes.

Preferably the leukocyte cells are lung derived monocytes.

Preferably the leukocyte cells are U937 cells.

Preferably the alveolar macrophage-like cells are U937 cells differentiated with PMA (phorbol-12-myristate-13-acetate) or with 1, 25 dihydroxyvitamin D3, most preferably differentiated with PMA.

Preferably all cells are immortalised mammalian cell lines, which are cells more phenotypically and functionally stable than primary cells and are more preferably immortalised human cell lines.

Preferably the alveolar type I epithelial cells are configured to form tight junctions and a polarised cell layer. This represents as close as is possible the barrier present to transport of inhaled chemicals/particles in the small airways/alveolus. This property is essential to be able to determine the systemic toxicity, biological response, therapeutic response, pharmacological response and potential absorption of molecules from the lungs into the body for the best prediction of toxicity/therapeutic effectiveness.

Preferably the alveolar macrophage-like cells are configured to participate to defence mechanisms by ingesting foreign materials by phagocytosis. This represents the functionality of the cells in vivo. This property indicates their ability to respond to chemical/particulate stimuli and the downstream signalling associated with the response. This functionality provides the best prediction of inflammatory responses and associated toxicity.

Preferably the alveolar type I epithelial cells are hAELVi alveolar type I epithelial cells and the alveolar macrophage-like cells are U937 cells, differentiated with PMA. This combination represents the two essential cell types present in the alveoli which provide the first responses to inhaled chemicals/particulates. Furthermore, it is established that alveolar macrophages and alveolar epithelial cell cross-talk is one of the key determinants in cascading inflammatory responses in the airways. Modelling the interaction between the cell types involved in the primary response to an inhaled compound provides a platform to determine the downstream response pathways and determine whether an adaptive/adverse response to an inhaled stimulus would be initiated.

Compared with the co-culture model of Kletting et al., the present model utilises U937 cells instead of THP-1 cells for the alveolar macrophage-like component. There are no human alveolar macrophage cell lines currently in existence, however the U937 cell line isolated from a human pleural effusion is a monocytic cell line originating from the lung with the capacity to most closely resemble the alveolar macrophage rather than the THP-1 cells which are from a blood monocyte population. Lung-derived macrophages are from a different lineage to blood monocyte-derived macrophages and hence they possess different characteristics and functionalities. Therefore, the use of U937 cells in the present model more precisely mimics the in vivo situation and provides the closest representation of an alveolar macrophage-like from a co-culture cell line model.

Compared with other U937 co-culture models, the present invention uses alveolar type I epithelial cells or a combination of both alveolar type I and type II epithelial cells rather than alveolar type II cells (e.g. A549) on their own. Alveolar type I epithelial cells comprise approximately 90% of the epithelial cell surface of the alveolus. Therefore, the present model mimics more precisely the in vivo situation where alveolar type I epithelial cells form a tight monolayer of cells in the alveolus and constitute the primary cell barrier to permeation of substances between the airspace and blood supply in the alveolus.

In one alternative the porous membrane separating the first or apical and second or basolateral compartments is a Transwell® or Snapwell® insert. Advantageously, the cell types are provided in different compartments (allowing the diffusion of chemical mediators between the cells and with the potential for migration through the porous membrane to more precisely mimic the in vitro conditions) making analysis of the responses of each cell population easier to assess and attribute more specific functional determination of response. For example, the alveolar type I epithelial cells or the combination of both alveolar type I and type II epithelial cells can respond to biochemical signals released by the alveolar macrophage-like cells and vice versa.

The three-dimensional in vitro alveolar lung model of the second and third aspects of the present invention finds interesting applications, in particular:
Assessing the toxicity of inhalable products such as particles or molecules on the alveolar barrier of the lungs;
Assessing the biological responses of inhalable products such as particles or molecules on the alveolar barrier of the lungs;
Assessing the pharmacological responses of inhalable products such as particles or molecules on the alveolar barrier of the lungs; and
Assessing the permeation of inhalable products such as particles or molecules across the alveolar barrier of the lungs.

According to a fourth aspect of the present invention there is provided a method of using the three-dimensional in vitro alveolar lung model of the second or third aspects of the present invention for assessing the response of a product on the alveolar barrier of lungs.

Preferably the method comprises the steps of:
a) exposing the product to be tested on the first or apical compartment of the three-dimensional model;
b) image analysis techniques to evaluate morphological characteristics (for example parameters including but not limited to cell size, cell shape, vacuole characteristics, organelle characteristics);
c) assessment of barrier function of the alveolar type I epithelial cells or the combination of both alveolar type I and type II epithelial cells (for example parameters including but not limited to transepithelial electrical resistance (TEER), paracellular permeability); and
d) further biological endpoints including but not limited to genotoxicity, biochemical markers of apoptosis, proteomics, transcriptomics, metabolic activation, cell membrane integrity may also be measured.

Alternatively, the method comprises the steps of:
a) exposing the product to be tested on the second or basolateral compartment of the three-dimensional model;
b) image analysis techniques to evaluate morphological characteristics (for example parameters including but not limited to cell size, cell shape, vacuole characteristics, organelle characteristics);
c) assessment of barrier function of the alveolar type I epithelial cells or the combination of both alveolar type I and type II epithelial cells (for example parameters including but not limited to transepithelial electrical resistance (TEER), paracellular permeability); and
d) further biological endpoints including but not limited to genotoxicity, biochemical markers of apoptosis, proteomics, transcriptomics, metabolic activation, cell membrane integrity may also be measured.

This allows for the testing of both inhalable products and also products taken orally or intravenously and by other delivery routes.

In one alternative the response is a toxicological response. In another alterative the response is an inflammatory response. In another alterative the response is a biological response. In another alterative the response is a pharmacological response. In another alternative the response is a biochemical response.

The product specifically includes particles and compounds.

According to a fifth aspect of the present invention there is provided the use of the three-dimensional in vitro alveolar lung model of the second or third aspects of the present invention for determining and/or predicting and/or inhibiting a response of a product on the alveolar barrier of lungs.

Preferably the use comprises the steps of:
a) exposing the product to be tested on the first or apical compartment of the three-dimensional model;
b) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
c) assessing markers for alveolar inflammation to be measured by flow cytometry or other biological assay (including but not limited to INF-gamma, TNF-alpha, IL-12, CXCL9-11, IL-8, IL-6, GM-CSF).

Alternatively, the use comprises the steps of:
a) exposing the product to be tested on the second or basolateral compartment of the three-dimensional model;
b) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
c) assessing markers for alveolar inflammation to be measured by flow cytometry or other biological assay (including but not limited to INF-gamma, TNF-alpha, IL-12, CXCL9-11, IL-8, IL-6, GM-CSF).

This allows for the testing of both inhalable products and also products taken orally or intravenously and by other delivery routes.

In one alternative the response is a toxicological response. In another alterative the response is an inflammatory response. In another alterative the response is a biological response. In another alterative the response is a pharmacological response. In another alternative the response is a biochemical response.

The product specifically includes particles and compounds.

According to a sixth aspect of the present invention there is provided a method for determining and/or predicting and/or inhibiting a response of a product on the alveolar barrier of lungs.

Preferably the method comprises the steps of:
a) exposing the product to be tested on the first or apical compartment of the three-dimensional model of the second or third aspects of the present invention; b) image analysis techniques to evaluate morphological characteristics (for example parameters including but not limited to as cell size, cell shape, vacuole characteristics, organelle characteristics);
c) assessment of barrier function of the alveolar epithelial component (for example parameters including but not limited to transepithelial electrical resistance (TEER), paracellular permeability);
d) further biological endpoints including but not limited to genotoxicity, biochemical markers of apoptosis, proteomics, transcriptomics, metabolic activation, macrophage (or cell) migration, cell membrane integrity may also be measured;

e) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
f) assessing markers for alveolar inflammation to be measured by flow cytometry or other biological assay (including but not limited to INF-gamma, TNF-alpha, IL-12, CXCL9-11, IL-8, IL-6, GM-CSF).

Alternatively, the method comprises the steps of:
a) exposing the product to be tested on the second or basolateral compartment of the three-dimensional model of the second or third aspects of the present invention;
b) image analysis techniques to evaluate morphological characteristics (for example parameters including but not limited to as cell size, cell shape, vacuole characteristics, organelle characteristics);
c) assessment of barrier function of the alveolar epithelial component (for example parameters including but not limited to transepithelial electrical resistance (TEER), paracellular permeability);
d) further biological endpoints including but not limited to genotoxicity, biochemical markers of apoptosis, proteomics, transcriptomics, metabolic activation, macrophage (or cell) migration, cell membrane integrity may also be measured;
e) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
f) assessing markers for alveolar inflammation to be measured by flow cytometry or other biological assay (including but not limited to INF-gamma, TNF-alpha, IL-12, CXCL9-11, IL-8, IL-6, GM-CSF).

This allows for the testing of both inhalable products and also products taken orally or intravenously and by other delivery routes.

In one alternative the response is a toxicological response. In another alterative the response is an inflammatory response. In another alterative the response is a biological response. In another alterative the response is a pharmacological response. In another alternative the response is a biochemical response.

Preferably the markers for the response include, but are not limited to, cell death, altered cell metabolism, initiation of apoptotic or other cell death pathways, compromised cell membrane integrity, altered cell biochemistry and altered cellular morphology.

Preferably the method includes the measurement of further biological endpoints comprising release of interleukins, genotoxicity, biomarkers of sensitization, proteomics, transcriptomics and metabolic activation.

The product specifically includes particles and compounds.

According to a seventh aspect of the present invention there is provided a method of using the three-dimensional in vitro alveolar lung model of the second or third aspects of the present invention for assessing a product.

Preferably the method is for assessing the fate of the product in the alveolar environment in the lungs.

Preferably the method comprises the steps of:
a) exposing the product on the first or apical compartment of the three-dimensional model;
b) assessing the concentration of the product and product metabolites within the model by an appropriate analytical tool (for example including but not limited to fluorescence, radiochemistry, LC-MS, HPLC);
c) assessing the localisation of the product and product metabolites within the model by an appropriate analytical tool (for example including but not limited to fluorescence microscopy, radiochemistry, image flow cytometry, SEM, TEM); and
d) assessing the physical characteristics (e.g. agglomeration) of the product within the model by an appropriate analytical tool (for example including but not limited to microscopy, SEM, TEM).

According to an eighth aspect of the present invention there is provided a kit of parts for creating a three-dimensional in vitro alveolar airway model according to the second or third aspects of the present invention comprising:
alveolar type I cells or a combination of both alveolar type I and type II epithelial cells;
alveolar macrophage-like cells;
cell culture medium;
cell culture supplements;
culture vessel; and
assembly instructions.

The three-dimensional in vitro alveolar lung model of the invention presents the following main advantages:
Presence of alveolar macrophage-like cells originating from leukocyte cells being monocyte cells derived from the human lung which possesses closer morphology and functionality to the in vivo situation than co-cultures which utilise monocyte derived macrophages from blood (e.g. THP-1 cells);
Presence of an alveolar type I epithelial cell line with functional barrier properties permitting the assessment of particle/molecule uptake in comparison with co-cultures which utilise A549 cells (alveolar type II epithelial cell line, no tight junctions);
Possibility of exposure at the air-liquid interface (ALI) using gases, vapours, aerosolised particles, liquids or powders as materials to be tested;
Possibility of testing particles and molecules for two aspects: inflammatory potential and permeation potential, contrary to other models which can only be used for inflammatory potential;
Possibility of measuring a multitude of biological endpoints (e.g. cell health, cell morphology, release of interleukins/cytokines, genotoxicity, biomarkers of inflammation, proteomics, transcriptomics, metabolic activation, intracellular cell signalling pathways, lipid profiles etc.).
Easy separation of the model to isolate each cell type after experiments or at any required time point to determine the response/viability/impact of each cell type separately as well as in combination.

### BRIEF DESCRIPTION OF THE DRAWINGS

Other aspects and features of the present invention will become apparent to those ordinarily skilled in the art upon review of the following description of specific embodiments of the invention in conjunction with the accompanying figures.

FIG. 1 illustrates the generation and construct methodology for T I model which is an optimised co-culture model of human alveolar type I epithelial cells (hAELVi) and differentiated U937 cells (MØ) where FIG. 1A illustrates U937 cells cultured on the bottom of the basolateral compartment and FIG. 1B illustrates the U937 cells cultured on the underside of the porous membrane;

FIG. 2 illustrates the generation and construct methodology for T II model which is an optimised co-culture model of human alveolar type II epithelial cells (A549) and differentiated U937 cells (MØ);

FIGS. 3A to 3E illustrate the optimisation of differentiation process for U937 cells to alveolar macrophage-like cells—assessment of CD 11a, CD 11b, CD 14, CD6, CD206 with different PMA exposure protocols and in comparison with primary human alveolar macrophages;

FIG. 4 illustrates the optimisation of differentiation process for U937 cells to alveolar macrophage-like cells—Phagocytic activity of PMA treated U937 cells and primary alveolar macrophage cells;

FIG. 5 illustrates the optimisation of differentiation process for U937 cells to alveolar macrophage-like cells—Morphology of U937 cells with exposure to PMA;

FIGS. 6A and 6B illustrate the optimisation of differentiation process for U937 cells to alveolar macrophage-like cells—Long-term proliferation of PMA treated U937 cells;

FIGS. 7A, 7B and 7C illustrate the maintenance of functionality of U937 cells after differentiation to alveolar macrophage-like cells with PMA where the fluorescence intensity indicates the presence of phospholipids in response to induction of phospholipidosis with amiodarone;

FIGS. 8A and 8B illustrate a comparison of epithelial cell mediated cytotoxicity when cultured on 3D Transwell® inserts to a 2D platform;

FIGS. 9A and 9B illustrate the impact of seeding density on TEER profiles of hAELVi and A549 cells on 0.4 μm Transwell® inserts for 20 days;

FIGS. 10A and 10B illustrate LDH detection of hAELVi cell cytotoxicity for liquid-liquid culture (LLC) cells at varying cell densities on 0.4 μm Transwell® inserts for up to 20 days;

FIGS. 11A and 11B illustrate LDH detection of A549 cell cytotoxicity for LLC cells at varying cell densities on 0.4 μm Transwell® inserts for up to 20 days;

FIGS. 12A and 12B illustrate the impact of seeding density of viability profiles of alveolar type I and type II epithelial cells;

FIGS. 13A and 13B illustrate the impact of different medium compositions on hAELVi cells, cultured in 96 well plate under LCC;

FIGS. 14A and 14B illustrate the viability and LDH release of A549 (B) cells cultured in different mediums for 20 days.

FIGS. 15A and 15B illustrate epithelial cell TEER profiles cultivated at air liquid interface (ALI) and under LLC conditions;

FIGS. 16A and 16B illustrate the impact of macrophages on barrier function of epithelial cells;

FIGS. 17A to 17D illustrate CD marker expressions in lipopolysaccharide (LPS) (LPS is a chemical which induces inflammation) stimulated and non-stimulated alveoli models;

FIG. 18 illustrates the human cytokine profile of lower airway for ALI cell models;

FIGS. 19A to 19C illustrate the human cytokine profile of lower airway T I models;

FIGS. 20A to 20C illustrate the human cytokine profile of lower airway T II models;

FIGS. 21A to 21C illustrate the human cytokine profile of lower airway; T I &T I I models in the presence of LPS;

FIG. 22 illustrates a table of the human cytokine profile of lower airway of mono-culture and co-culture models in LPS;

FIG. 23 illustrates a comparison of macrophages in mono-culture and co-cultures phagocytosis of microspheres;

FIGS. 24A and 24B illustrate phagocytic activity of differentiated U937 cells (MØ) in mono-culture and co-cultures in the presence of LPS;

FIGS. 25A and 25B illustrate comparison of mono-culture and co-culture construction on cell health with and without LPS;

FIGS. 26A and 26B illustrate the impact of LPS on barrier properties of alveolar type I and type II epithelial cells in co-culture;

FIG. 27 illustrates a visual example of model with hAELVi-PMA-differentiated U937 cells at the ALI;

FIG. 28A illustrates the generation and construct methodology for a mixed population T I and T II model;

FIG. 28B illustrates the generation and construct methodology for a multi-layered population T I and T II model;

FIG. 29 illustrates the mixed population of T I and T II epithelial cells cultured at a ratio of A (1:1),B (2:1),C (10:1), D (20:1) hAELVi cells:A549 cells;

FIG. 30 illustrates viability data of hAELVi and A549 cells cultured at different ratios in a 96 well plate;

FIG. 31 illustrates viability data of hAELVi and A549 co-cultured in Transwell® inserts;

FIG. 32 illustrates the presence of tight junctions and a functional, polarised epithelial cell layer;

FIG. 33 illustrates the TEER values of TI/TII and differentiated U937 cells cultured in different co-culture set ups, cultured under LLC;

FIG. 34 illustrates surfactant proteinc(SPC) production from A549 cells grown as a layer on top of hAELVi cells in ALI at a 10:1 ratio (hAELVi:A549);

FIG. 35 illustrates the functionality of response of alveolar macrophage-like cells to induction of phospholipidosis; and

FIG. 36 illustrates extent of immune response using IL-8 secretion.

### DETAILED DESCRIPTION

The present invention provides for a method for preparing a three-dimensional in vitro alveolar lung model comprising a culture well provided with a membrane configured to separate the well into a first compartment and a second compartment, wherein alveolar type I epithelial cells are provided in the first compartment and alveolar macrophage-like cells are provided in the second compartment, wherein the membrane has a first side configured to form a wall of the first compartment and a second side configured to form a wall of the second compartment.

The present invention also provides for a three-dimensional in vitro alveolar lung model comprising a culture well provided with a membrane configured to separate the culture well into a first compartment and a second compartment, wherein the membrane has a first side configured form a wall of the first compartment and a second side configured to form a wall of the second compartment, wherein alveolar type I epithelial cells are provided in the first compartment and alveolar macrophage-like cells are provided in the second compartment.

In an embodiment of the invention the first compartment is configured to be exposed to an air-liquid interface (ALI) and the second compartment configured to be submerged in a culture medium. In an alternative the second compartment is configured to be exposed to an air-liquid interface (ALI) and the first compartment configured to be submerged in a culture medium. In a further alternative both the first and second compartments are configured to be submerged in a culture medium. Preferably where the second compartment is exposed to the ALI and the first compartment is submerged the cells are provided on the reverse side of the membrane of the second compartment.

In an embodiment of the invention the first compartment comprises an apical compartment and the second compartment comprises a basolateral compartment.

In an embodiment of the invention first side of the membrane is an apical side and the second side of the membrane is a basolateral side.

In an embodiment of the invention the alveolar type I epithelial cells are hAELVi cells.

In an alternative embodiment of the invention a combination of both alveolar type I epithelial cells and alveolar type II epithelial cells are provided in the first compartment, preferably a combination of hAELVi cells and A549 cells.

In an embodiment of the invention the method comprises preparing a co-culture of a) alveolar type I epithelial cells or combination of alveolar type I and type II epithelial cells and b) alveolar macrophage-like cells.

The first step in the preparation of the co-culture is to prepare the alveolar type I epithelial cells or combination of alveolar type I and type II epithelial cells.

This includes the preparation of the medium for the alveolar type I epithelial cells or combination of alveolar type I and type II epithelial cells (first culture medium), an example of the this medium (hAELVi medium) preparation is set out below:
Remove 60 mL of huAEC medium (InSCREENeX);

http://www.inscreenex.de/products/human-immortalized-cell-lines/alveolar-epithelial-cells-hu.html) from a new bottle (500 mL)
Add the following huAEC basal supplements (InSCREENeX):
   bovine pituitary extract 2 mL
   insulin 0.5 mL
   gentamicin sulfate and amphotericin (GA-1000) 0.5 mL
   retinoic acid 0.5 mL
   bovine serum albumin-fatty acid free (BSA-FAF) 5 mL
   transferrin 0.5 mL
   triiodo-L-thyronine (T3) 0.5 mL
   epinephrine 0.5 mL
   recombinant human epidermal growth factor (rhEGF) 0.5 mL do not add the 0.5 mL of hydorcortisone
Add 25 mL FBS
Add 5 mL penicillin/streptomycin

The second step in the preparation of the co-culture is to prepare the alveolar macrophage-like cells.

In one alternative the alveolar macrophage-like cells are differentiated U937 cells.

This includes the preparation of the medium for the alveolar macrophage-like cells (second culture medium), an example of the this medium (U937 medium) preparation is set out below:
Remove 60 mL of RPMI medium from a new bottle (500 mL)
Add 50 mL of FBS (10% v/v final concentration)
Add 5 mL of 200 mM L-glutamine (2 mM or 1% v/v final concentration)
Add 5 mL of penicillin/streptomycin (1% final concentration).

The U937 medium is also used as the co-culture medium in the model.

The co-culture according to an embodiment of the invention is prepared using the following step sequence:
i) seeding the first or apical side of the membrane with the alveolar type I epithelial cells or combination of alveolar type I and type II epithelial cells;
ii) introducing the membrane into a first culture well such that the alveolar type I epithelial cells or combination of alveolar type I and type II epithelial cells are present in the first or apical compartment at the air-liquid interface (ALI);
iii) introducing the first culture medium into the first culture well;
iv) culturing the alveolar type I epithelial cells or combination of alveolar type I and type II epithelial cells;
v) seeding a second culture well with leukocyte cells in the second culture medium;
vi) differentiating the leukocyte cells to alveolar macrophage-like cells; and
vii) removing the membrane with the cultured alveolar type I epithelial cells or combination of alveolar type I and type II epithelial cells from the first culture well and introducing the membrane with the cultured alveolar type I epithelial cells or combination of alveolar type I and type II epithelial cells into the second culture medium of the second culture well such that the alveolar macrophage-like cells, present in the second or basolateral compartment are submerged in the second culture medium, and that the alveolar type I epithelial cells or combination of alveolar type I and type II epithelial cells are present in the first or apical compartment at the ALI.

In an alternative co-culture is prepared using the following step sequence:
i) seeding the first side of the membrane with the alveolar type I epithelial cells or combination of alveolar type I and type II epithelial cells;
ii) seeding the second side of the membrane with leukocyte cells;
iii) introducing a second culture medium into culture well;
iv) introducing the membrane into a culture well such that the alveolar type I epithelial cells or combination of alveolar type I and type II epithelial cells are present in the first compartment preferably at the air-liquid interface (ALI); v) introducing a first culture medium into culture well;
vi) culturing the alveolar type I epithelial cells or combination of alveolar type I and type II epithelial cells;
vii) differentiating the leukocyte cells to alveolar macrophage-like cells.

Seeding is defined as introducing a defined amount (volume or cell number) of a cell suspension into a container (such as the culture cell) or onto a surface (such as the membrane).

The first or apical side of the membrane is seeded with between 1×10⁴ and 5×10⁵ alveolar type I epithelial cells or combination of alveolar type I and type II epithelial cells/cm², more preferably 1×10⁵ alveolar type I epithelial cells or combination of alveolar type I and type II epithelial cells/cm².

The first or apical side of the membrane, which is seeded with the alveolar type I epithelial cells or combination of alveolar type I and type II epithelial cells, is raised to the air-liquid interface after seeding.

The alveolar type I epithelial cells or combination of alveolar type I and type II epithelial cells are cultured at the air liquid interface.

The culture cell or second side or basolateral side of the membrane is seeded with 1.75×10⁵ lymphocyte cells/cm².

The alveolar type I epithelial cells or combination of alveolar type I and type II epithelial cells are cultured for between 4-28 days, preferably for 10 days.

Culturing is defined as the maintenance or growth of cells in controlled conditions outside of their native environment.

The method further comprises differentiating the leukocyte cells to alveolar macrophage-like cells.

Differentiating is defined as the processes applied to a cell which enable it to undergo changes in gene expression to become a more specific type of cell.

The leukocyte cells are differentiated to alveolar macrophage-like cells with PMA (phorbol-12-myristate-13-acetate) or with 1, 25 dihydroxyvitamin D3, most preferably differentiated with PMA.

The differentiation is performed over several days: preferably 1-7 days and more preferably 3 days.

The alveolar type I epithelial cells or combination of alveolar type I and type II epithelial cells are seeded on to the membrane in 0.5 mL of hAELVi culture medium.

The leukocyte cells are seeded on to the bottom of a 24-well plate in 1 mL of U937 culture medium.

In an embodiment of the invention the membrane comprises a porous membrane.

The porous membrane is configured for potential migration of the alveolar macrophage-like cells between the second and first compartments, preferably between the basolateral compartment and the apical compartment.

The porous membrane is provided with a plurality of pours, preferably the pours are between 0.4-10 μm in diameter, more preferably 0.4 and 8 μm in diameter, and even more preferably between 0.4 and 3 μm in diameter.

Optionally, a perfusion system is provided to allow for circulation of the first and/or second culture mediums, in one alternative the perfusion system is an external perfusion system. In an embodiment of the invention the membrane is pre-treated for optimal cell growth.

In an embodiment of the invention the pre-treatment comprises a coating or coating methodology.

In an embodiment of the invention the coating is provided on the first side of the membrane, preferably the coating is provided on the apical side of the membrane, preferably the coating is provided on the growth surface of the alveolar type I epithelial cells or combination of alveolar type I and type II epithelial cells.

In an embodiment of the invention the coating comprises a biological and/or synthetic polymer.

In an embodiment of the invention the coating comprises collagen, gelatin, laminin fibronectin, poly-L-lysine or serum.

In an embodiment of the invention the coating is selected from collagen, gelatin, laminin fibronectin, poly-L-lysine or serum.

In an embodiment of the invention the coating is configured to optimise cell attachment, proliferation and function for the alveolar type I cells or combination of alveolar type I and type II epithelial cells to exhibit morphology and functionality that most closely resembles that of alveolar type I cells or combination of alveolar type I and type II epithelial cells in their native environment.

Step v) takes place about 7-14 days after the seeding of the alveolar type I epithelial cells or combination of alveolar type I and type II epithelial cells and after about 24 hours after differentiation of the alveolar macrophage-like cells.

Ideally all cells are immortalised mammalian cell lines, which are cells more phenotypically and functionally stable than primary cells and are more preferably immortalised human cell lines.

The alveolar type I epithelial cells or combination of alveolar type I and type II epithelial cells are configured to form tight junctions and a polarised cell layer.

The alveolar macrophage-like cells are configured to participate in defence mechanisms by ingesting foreign materials by phagocytosis.

In one alternative the alveolar type I epithelial cells are hAELVi alveolar type I epithelial cells and the alveolar macrophage-like cells are U937 cells, differentiated with PMA.

In one alternative the membrane separating the first or apical and second or basolateral compartments is a porous membrane being a Transwell® or Snapwell® insert. Advantageously, the cell types are provided in different compartments (with the potential for migration through the porous membrane to more precisely mimic the in vitro conditions) making analysis of the responses of each cell population easier to assess and attribute more specific functional determination of response.

The culture medium is selected from one or more of DMEM, DMEM/F12 (50:50), RPMI, SAGM (Lonza), hAEC, MucilAir, SmallAir (Epithelix), huAEC (InScreenex) and preferably RPMI or huAEC.

An exemplary preparation of the three-dimensional in vitro alveolar lung model is set out below:

Day 0: Human Alveolar Epithelial Lentivirus immortalized (hAELVi) cells are seeded on the apical surface of cell culture inserts at a concentration of 1×10⁵ cells/cm². The basolateral chamber is also filled with hAELVi medium. Cells are incubated for 48 h at normal cell cultivation conditions (37° C., 5% v/v CO₂).

Day 2: hAELVi medium is removed from both apical and basolateral chambers and fresh hAELVi medium is added to the basolateral chamber only.

Day 4: hAELVi medium is removed from the chambers and fresh hAELVi medium is added to the basolateral chamber only.

Day 6: hAELVi medium is removed from the chambers and fresh hAELVi medium is added to the basolateral chamber only.

Day 8: hAELVi medium is removed from the chambers and fresh hAELVi medium is added to the basolateral chamber only.

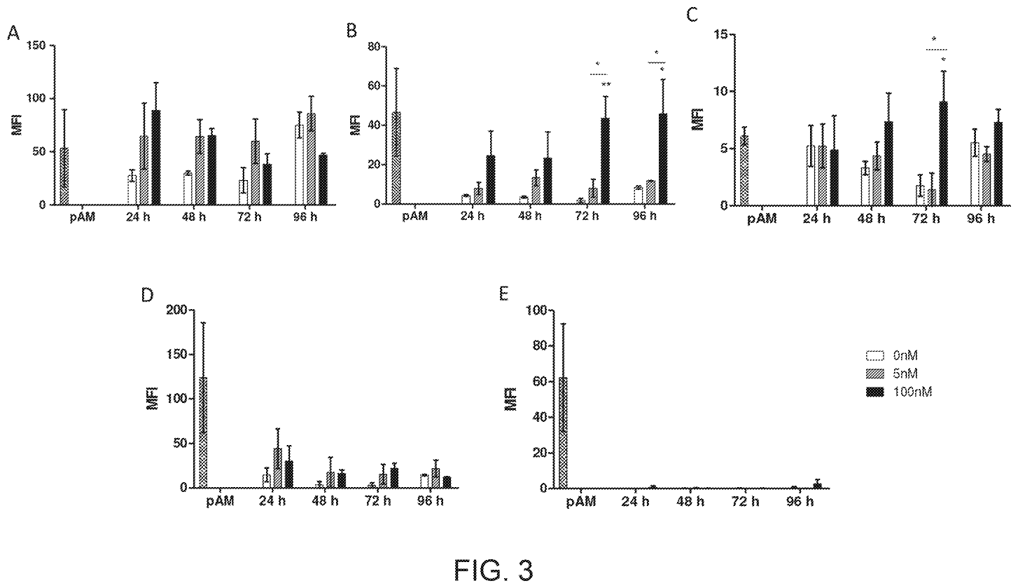

Day 8: U937 cells are differentiated with either PMA (phorbol-12-myristate-13-acetate) or with 1,25-dyhydroxyvitamin D3. In this example, U937 cells are seeded at a concentration of 1.75×10⁵ cells/cm² on to a well plate using U937 medium with 100 nM PMA (dissolved in DMSO; <1% v/v). Cells are incubated for 72 h at normal cell cultivation conditions (37° C., 5% v/v CO₂) for differentiation into mature alveolar macrophage-like cells as validated (FIGS. 3-6).

Day 10: hAELVi medium is removed from the chambers and fresh hAELVi medium is added to the basolateral chamber only.

Day 11: U937 medium is removed from the differentiated U937 cells and replenished with fresh U937 medium without PMA (24 h rest period).

Day 12: hAELVi medium is removed from the chambers. U937 medium is removed from PMA-differentiated U937 cells. Insert containing the hAELVi cells is placed into the well plate containing the U937 cells. Co-culture medium is added to the basolateral chamber of the co-culture and cells are incubated for 24 h at normal cell cultivation conditions (37° C., 5% v/v CO₂).

Day 13 onwards: Exposure of the system to the inhalable product (molecules or particles) to be tested on the model constructed as outlined above can be performed after 1 h but preferably 24-72 h after construction to allow for cell equilibration to new environment. Exposure may be through deposited particulates (e.g. VitroCell, NGI, TSI, ACI) or solubilised/suspended in cell culture medium and added to the apical and/or basolateral compartment(s).

The biological endpoints from 0-96 h after exposure depending on the expected time- and end—points can be performed.

Possible biological endpoints to be measured are for instance: cell morphology, cell viability, cytotoxicity, cell proliferation, cytokine secretion, macrophage activation, phagocytosis, TEER, staining for immune-cytochemistry or immune-fluorescence or RNA/protein extraction. This list is not limitative.

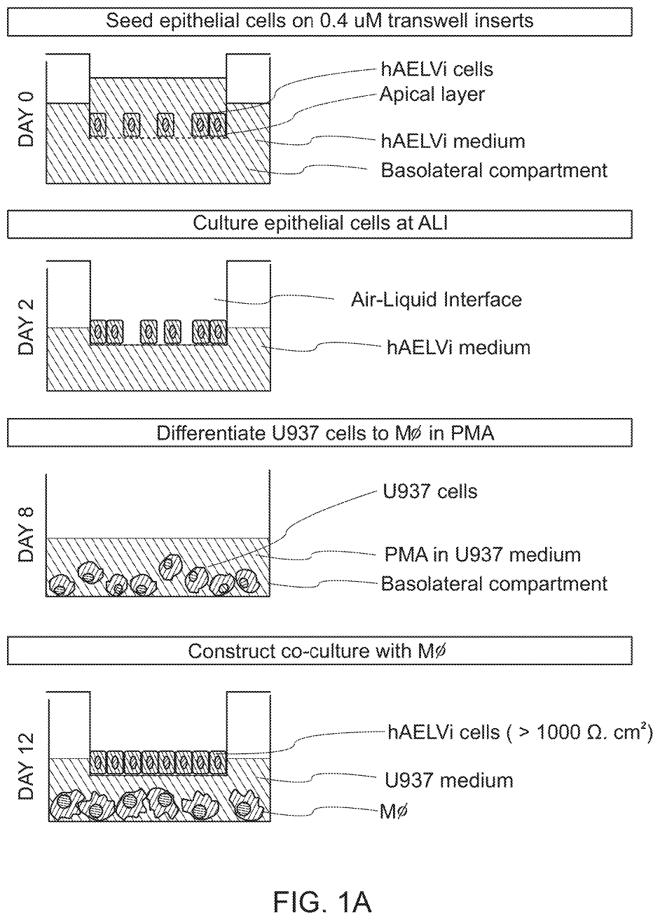

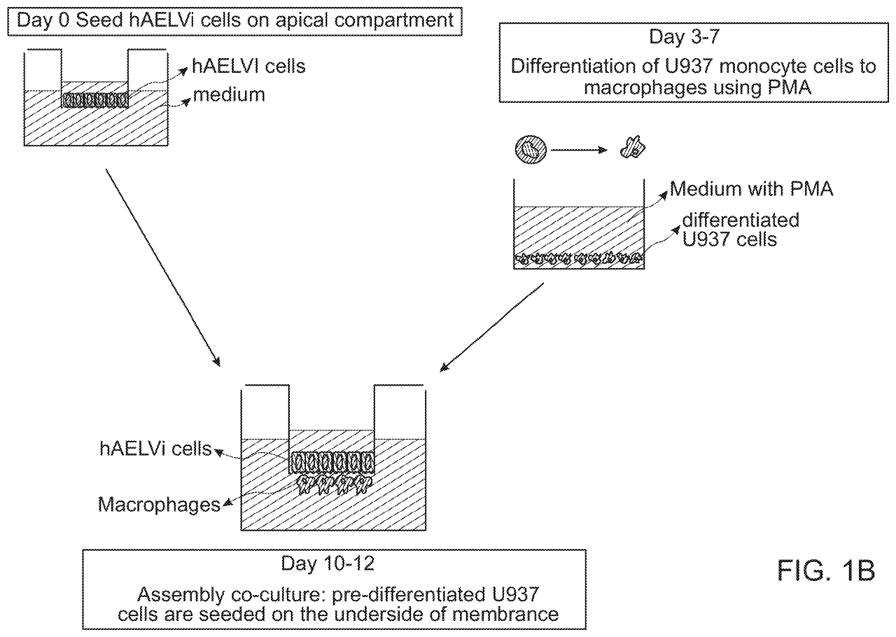

FIG. 1 illustrates the generation and construct methodology for T I model which is an optimised co-culture model of human alveolar type I epithelial cells (hAELVi) and differentiated U937 cells (MØ). The model was incubated for 24 h following construction.

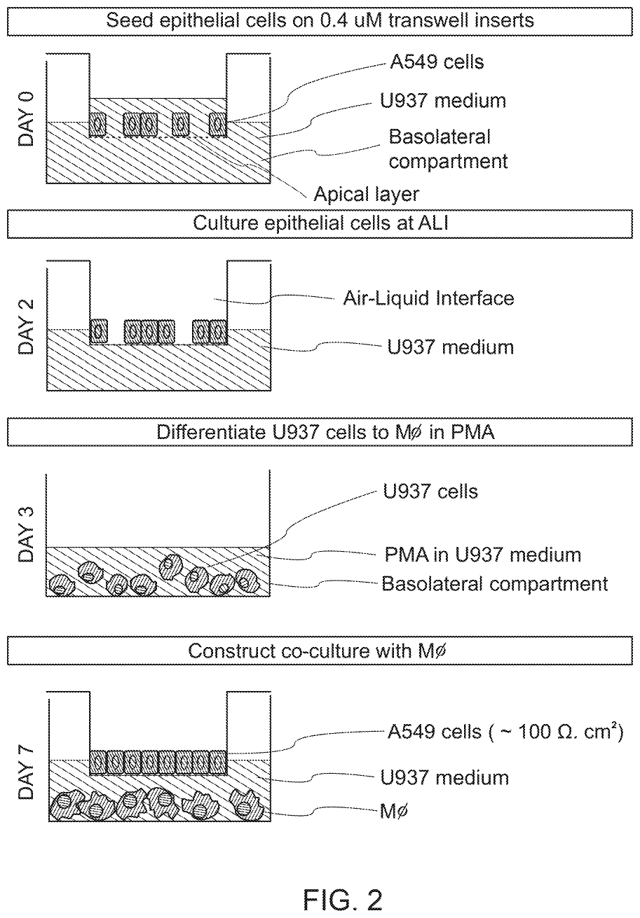

FIG. 2 illustrates the generation and construct methodology for T II model which is an optimised co-culture model of human alveolar type II epithelial cells (A549) and differentiated U937 cells (MØ). The model was incubated for 24 h following construction.

FIG. 26 illustrates a visual example of model with hAELVi-PMA-differentiated U937 cells at the ALI in use during the testing of compounds indicating where the compounds are added and where the response can be seen.

Experimental Data

Co-Culture Cultivation and Assembly

Alveolar-Like Macrophage Generation

Human monocytic U937 cells derived from pleural effusion were seeded at 5×10⁵ cells/mL in a 24 well plate or on the underside of a culture insert with 3.25×10⁵ cells per well with 100 nM phorbol 12-myristate 13-acetate (PMA) (dissolved in DMSO; <1% v/v) in complete RPMI medium (10% v/v FBS, 1% v/v penicillin-streptomycin, 2 mM L-glutamine). Cells were incubated for 72 h at 37° C., 5% v/v CO₂ in a humidified incubator to differentiate the cells to mature macrophages. Following PMA incubation, media was replaced with fresh CCM and incubated for a further 24 h rest period. After the 24 h rest phase, U937/MØ were ready for co-culture assembly. U937/MØ cells were prepared in line with when epithelial cells were ready, i.e. day 5 for A549 and day 10 for hAELVi cells.

The culturing of cells and model assembly are described for the A549 model and the hAELVi model as follows:

Macrophage Cultivation

Day (2): Seed U937 cells with 100 nM PMA.

Day (3): 24 h

Day (4): 48 h

Day (5): 72 h, change media

Day (6): 24 h rest period complete. Add epithelial cells with fresh medium (RPMI).

Alveolar Epithelial Cell Cultivation

Human alveolar epithelial cell lines A549 and hAELVi (hAELVi—human Alveolar Epithelial Lentivirus immortalized) were cultivated onto coated (coating solution, InScreenEx, Germany), T75 flasks or Transwell® membranes with a pore size either 0.4 μm, 3.0 μm and growth areas of 0.33 cm² (Corning: 3470; 3472). Cells were seeded at 1×10⁵ cells/cm² (3.3×10⁴ cells per tranwell) in either complete RPMI, supplemented with 2 mM L-glutamine, and 10% v/v FBS for A549 or small airway growth medium (HuAEC medium, basal supplement) containing 5% FBS for hAELVi cells. Two days after seeding, the seeded Transwell® filters were divided into two groups, one for culturing under LLC and the other at ALI.

To set up ALI cultures, the cells were seeded under LLC, i.e. 100 μL apical/600 μL basolateral; after two days in culture the medium was then completely aspirated, and the cells were further fed from the basolateral compartment i.e. 600 μL basolateral only, as described by Kletting (Kletting, 2016). The medium was changed every second day. To characterise and compare cell growth of A549 and hAELVi cells under both LLC and ALI culture conditions. TEER measurements were performed for up to 20 days.

A549 model

Day (0): A549 cells were seeded on transwell inserts in RPMI medium.

Day (2): Media was removed from the apical compartment for ALI and media in the basolateral compartment was replaced.

Day (4): Media change

Day (6): Media change

Day (7): A549 cells are confluent and healthy (determined by Viacount viability assay, lactate dehydrogenase (LDH) (LDH is an enzyme that indicates permeability of the cell membrane and indicator of cell death) for model assembly with U937/MØ

hAELVi Model

Day (0): hAELVi cells were seeded on transwell inserts in huAEC medium.

Day (2): Media was removed from the apical compartment for ALI and media in the basolateral compartment was replaced.

Day (4): Media change

Day (6): Media change

Day (8): Media change

Day (10): Media change

Day (12): hAELVi cells are confluent and healthy (determined by Viacount viability assay, LDH and TEER) for model assembly with U937/MØ. hAELVi cells with TEER reading of >1000Ω·cm² were used for co-culture model.

hAELVi and A549 Model (Layered)

Day (0): hAELVi cells were seeded on transwell inserts in huAEC medium.

Day (2): Media was removed from the apical compartment for ALI and media in the basolateral compartment was replaced.

Day (4): Media change

Day (6): Media change

Day (8): Media change

Day (10): Media change

Day (12/13)—On top of previously cultured hAELVi cells, type II A549 cells are seeded as a layer using a 10:1 ratio (hAELVi:A549 cells).

Day (14): Epithelial cells are confluent and healthy (determined by Viacount viability assay, LDH and TEER) for model assembly with U937/MØ. Epithelial cell layers with TEER reading of >1000 Ω·cm² were used for co-culture model.

hAELVi and A549 Model (Mixed)

Day (0): hAELVi and A549 cells a were seeded in combination at a ratio of 10:1 on transwell inserts in huAEC medium.

Day (2): Media was removed from the apical compartment for ALI and media in the basolateral compartment was replaced.

Day (4): Media change

Day (6): Media change

Day (8): Media change

Day (10): Media change

Day (12): Epithelial cells are confluent and healthy (determined by Viacount viability assay, LDH and TEER) for model assembly with U937/MØ. Epithelial cell layers with TEER reading of >1000Ω·cm² were used for co-culture model.

Assembly

Inserts were rinsed with RPMI prior to assembly with U937/MØ. Model was assembled with epithelial cells on the apical transwell membrane and U937/MØ on the basolateral/base of the well. Each model was incubated at normal cell cultivation conditions for 24 h before further testing, i.e. toxicity studies.

FIGS. 1 and 2 show a schematic overview of the cell culture methodology for constructing the alveolar epithelial-alveolar macrophage-like co-cultures.

FIG. 1 illustrates the generation and construct methodology for T I model which is an optimised co-culture model of human alveolar type I epithelial cells (hAELVi) and differentiated U937 cell (MØ) where 1A illustrates U937 cells cultured on the bottom of the basolateral compartment and 1B illustrates the U937 cells cultured on the underside of the porous membrane; FIG. 2 illustrates the generation and construct methodology for T II model which is an optimised co-culture model of human alveolar type II epithelial cells (A549) and differentiated U937 cell (MØ).

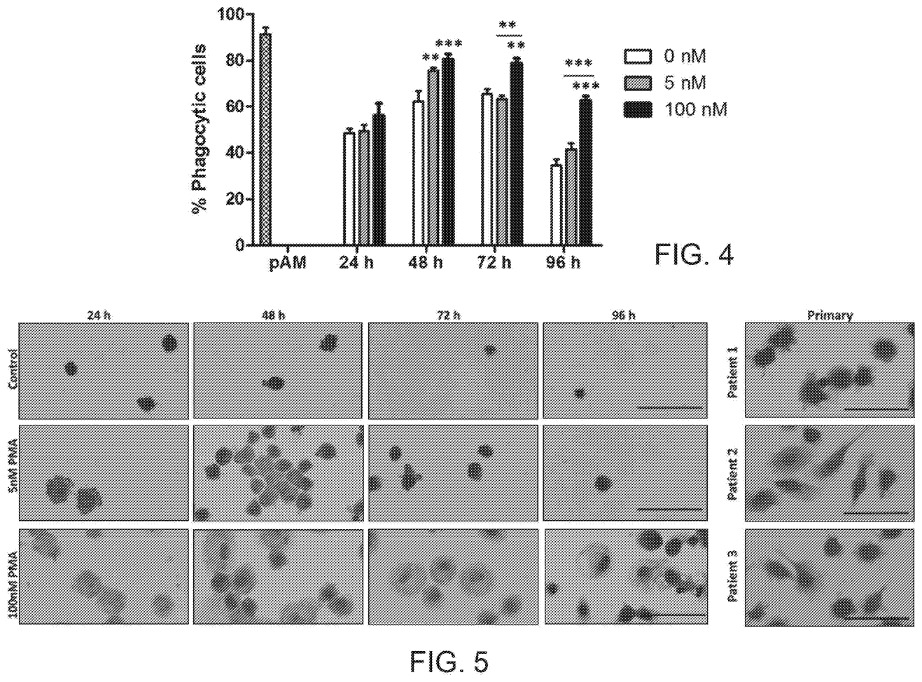

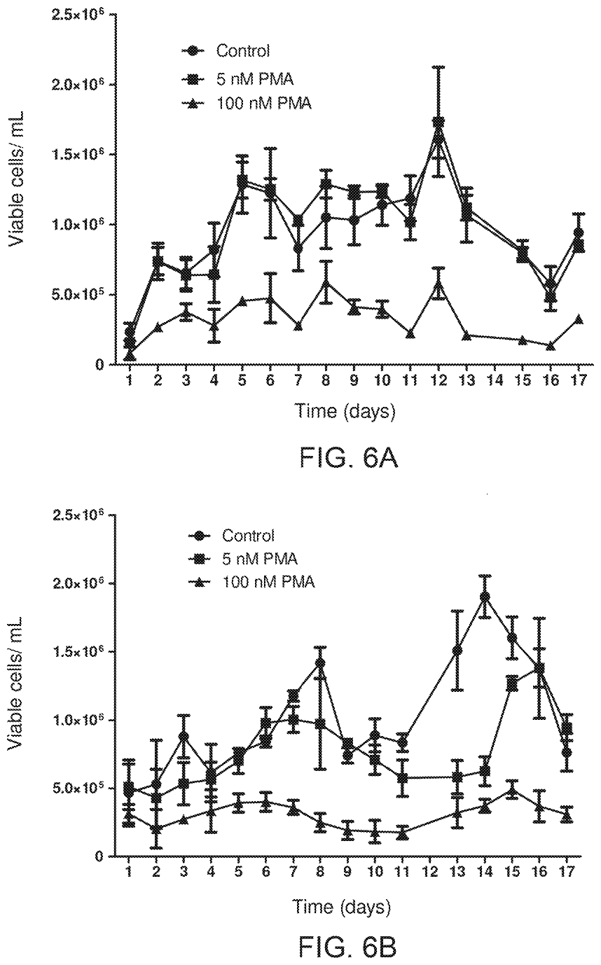

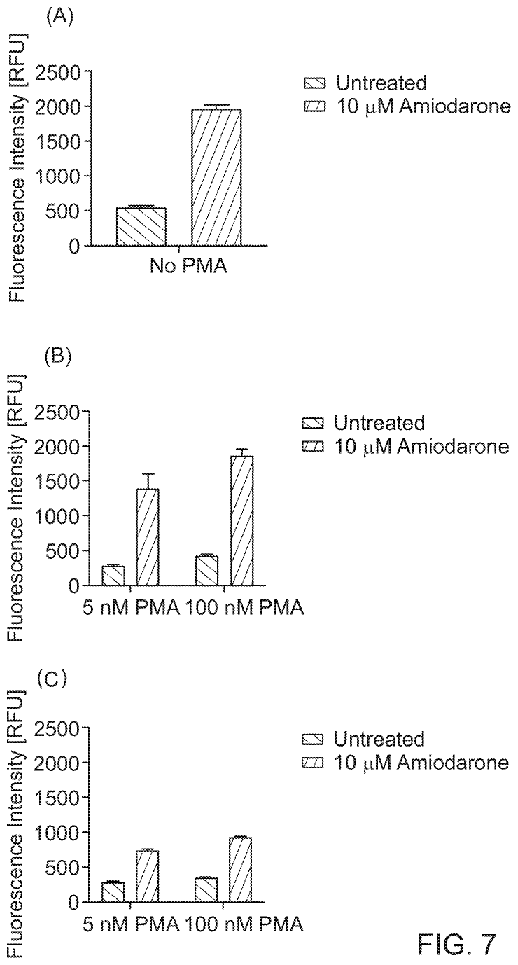

FIGS. 3 to 6 provide evidence to demonstrate that the PMA-differentiated in vitro alveolar macrophage-like cells of the present invention closely mimic in-vivo alveolar macrophages. The optimal PMA differentiation protocol was selected that generates the closest phenotype resembling primary human alveolar macrophages as possible determined by the presence of cell surface markers (FIG. 3), phagocytic ability (FIG. 4) morphology (FIG. 5), proliferation (FIG. 6) and that they maintain a response to amiodarone (established phospholipidosis) which is more representative of AM than undifferentiated monocytes (FIG. 7).

FIG. 3 illustrates the expression profiles of selected CD markers in PMA treated U937 cells and primary alveolar macrophages (pAM). CD markers are: (A) CD 11a, (B) CD 11b, (C) CD 14, (D) CD 36 and (E) CD 206. U937 cells treated with (0 nM, 5 nM, and 100 nM) PMA for 24 h to-96 h followed by a 24 h rest period in fresh culture medium. Expression of CD markers was measured by flow cytometry and are presented as median fluorescent intensity (MFI±SEM). A two way ANOVA was used to determine significance for each treatment group and Bonferroni post hoc test employed when * :p<0.05, ** : p<0.01 where n=12 of four cell passages for U937 cells. Human primary AMs were cultured, where n=3 patients. This demonstrates that the profile of cell surface markers selected to identify an alveolar macrophage phenotype were optimally expressed in PMA-differentiated U937 cells after 72 h and 100 nM exposure concentrations and were in line with the expression in primary human alveolar macrophages (A-C). CD36 and CD206 (D,E) are cell surface markers indicative of M2 polarised macrophages rather than the MØ population and expression of these marker is low as anticipated.

FIG. 4 illustrates the phagocytic activity of PMA treated U937 cells and primary alveolar macrophages (pAM). Percentage of phagocytic cells (mean±SEM) from PMA treated U937 cells. (n=9) for each time point for U937 cells and n=3 for primary cells. This demonstrates that the 72 h, 100 nM PMA differentiation protocol also resulted in cells with the same phagocytic functionality of primary human alveolar macrophages.

FIG. 5 illustrates the morphology of U937 cells with exposure to PMA. U937 cells were cultured on chamber slides with medium supplemented with either 0 nM, 5 nM or 100 nM PMA and incubated for 24 h, 48 h, 72 h and 96 h followed by a 24 h recovery phase in fresh medium. Non-adherent PMA naive cells required centrifuge preparation. Primary human alveolar macrophages were harvested for morphological comparison. The cells were photographed at ×400 magnification with an inverted light microscope. (50 um scale bar). This demonstrates that the 72 h, 100 nM PMA differentiation protocol also resulted in cells with the most similar morphology to primary human alveolar macrophages.

FIG. 6 illustrates the long-term proliferation of PMA treated U937 cells. Cells were seeded at 5×10⁵ cells/mL with no PMA (control), 5 nM or 100 nM PMA for 24 h (A) or 96 h (B) followed a 24 h rest period in fresh complete cell culture medium which was exchanged every 48 h. Cell proliferation was assessed using Guava ViaCount assay via flow cytometry for 10,000 events. Results shown are representative for two repeated experiments performed in triplicate. Viable cells/mL are shown as mean±SD. This demonstrates that U937 cells exposed to 100 nM PMA have reduced (24 h exposure) or no (96 h exposure) cell proliferation in line with primary human alveolar macrophages which do not proliferate in vivo.

FIG. 7 illustrates the maintenance of functionality of U937 cells after differentiation to alveolar macrophage-like cells with PMA. Cells were seeded at 5×10⁵ cells/mL with no PMA (control), 5 nM or 100 nM PMA for 24 h (A) or 72 h (B) followed a 24 h rest period in fresh complete cell culture medium. Phospholipidosis in the presence and absence of amiodarone 10 μM was assessed using flow cytometry using a LipidTOX green fluorescent stain to quantify extent of phospholipids per cell. Average mean fluorescence intensity of the LipidTOX fluorescent stain is expressed from is 5000 cells n=4. Fluorescence intensity indicates the presence of phospholipids in response to induction of phospholipidosis with amiodarone. This demonstrates cell functionality to amiodarone (established phospholipidosis) is maintained after differentiation.

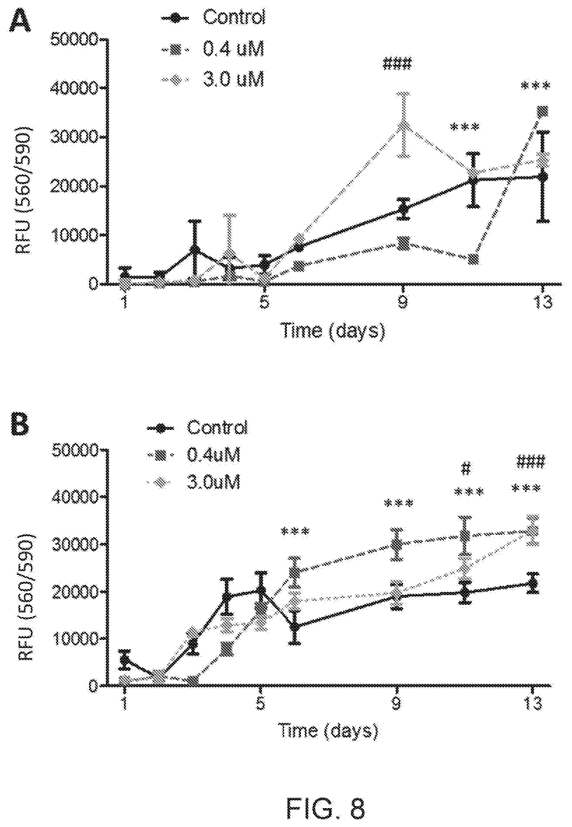

FIGS. 8 to 15 provide evidence to demonstrate the optimal culture environment (cell density, model substrate, composition of medium) to ensure epithelial cells retain suitable viability (number of viable cells, LDH release (cytotoxicity) and are as functionality relevant (TEER and formation of polarised cell layer for type I cells) to primary alveolar epithelial cells.

FIG. 8 illustrates a comparison of epithelial cell mediated cytotoxicity when cultured on 3D Transwell® inserts to a 2D platform. Alveolar epithelial cells: (A) hAELVi and (B) A549 were seeded (1×10⁵ cells/cm²) and cultured for 13 days in a 96 well plate (control), or a 0.4 μm or 3.0 μm Transwell® inserts (volume adjusted) to measure the LDH released into the supernatant in the apical compartments from the Transwell®. Data shown are the mean±SD of three Transwell® s. Data is considered significant for 0.4 μm (*) or 3.0 μm (#) p values where p<0.0001 (***), p<0.05 (*) vs control. This demonstrates the viability of both epithelial cell lines cultured on Transwell inserts with different pore sizes. The 0.4 um pore size is marginally more optimal for hAELVi cell viability but both are in-line with cell viability in a 2D system.

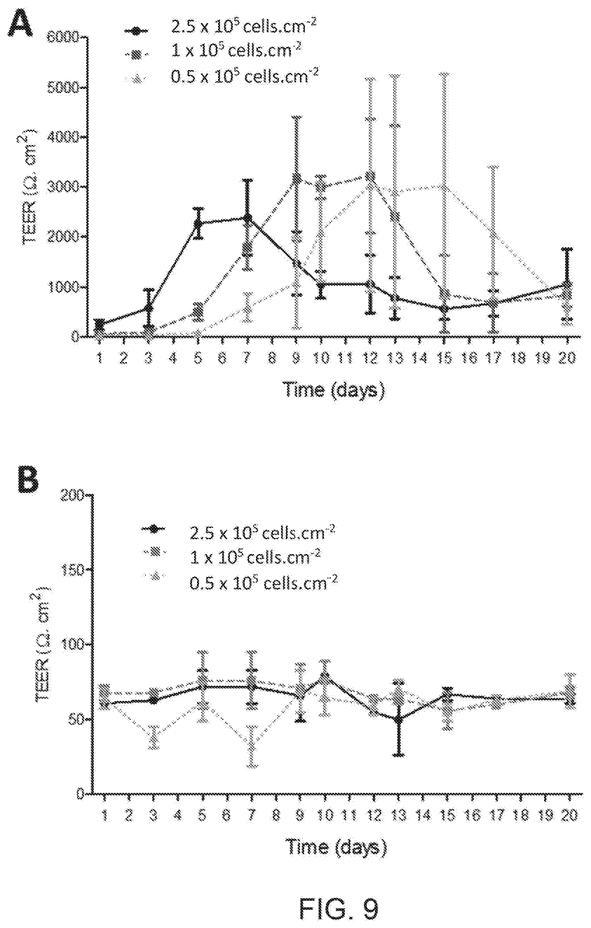

FIG. 9 illustrates the impact of seeding density on TEER profiles of hAELVi and A549 cells on 0.4 μm Transwell® inserts for 20 days. The TEER profiles for human alveolar epithelial cells (A) hAELVi and (B) A549 seeded at densities of 2.5×10⁵ cells/cm², 1×10⁵ cells/cm² and 0.5×10⁵ cells/cm² cultured on 24 well Transwell® inserts. Cell types were cultured for 20 days in submerged conditions with replenishment of media every 24 hours. Results were adjusted for the resistance of the filter and normalised to the area of the insert. Data are represented as mean±SD of 4-6 inserts. This demonstrates the optimal seeding density for hAELVi cells on the Transwell® insert was 1×10⁵ cells/cm² as these had an appropriate transepithelial electrical resistance in line with that reported in literature for primary alveolar epithelial cells between days 9-20 in culture. A549 cells (type II) as expected do not form polarised epithelial cell layers.

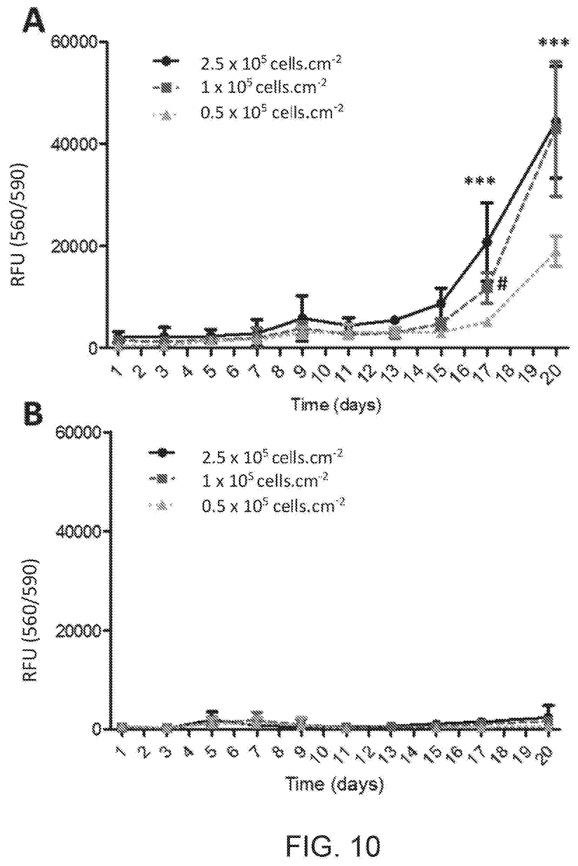

FIG. 10 illustrates LDH detection of hAELVi cell cytotoxicity for cells grown in submerged conditions at varying cell densities on 0.4 μm Transwell® inserts for up to 20 days. Cells were apically seeded in 200 μL volumes at cell densities of 2.5×10⁵ cells/cm², 1×10⁵ cells/cm² and 0.5×10⁵ cells/cm². Supernate samples (n=3) were collected from both apical (A) and basolateral (B) compartments from independent (n=4) Transwell® inserts of two cell passages. Data are expresses as mean±SD of each day's observation. Bonferroni post hoc test after two-way ANOVA where #:p<0.05, *** :pp<0.0001 is shown. This demonstrates cell viability alveolar type I epithelial cells in culture over the timeframe of model construction. Increasing LDH concentrations in the apical compartment indicate apical maturation of cells as occurs in vivo. Background/low levels of LDH in basolateral compartment indicate viable cell layer adjacent to porous membrane.

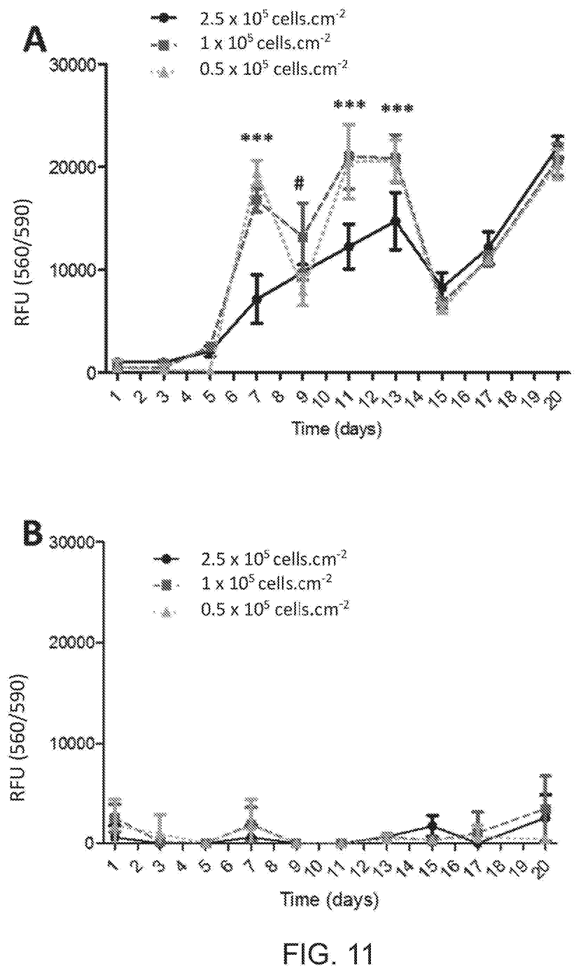

FIG. 11 illustrates LDH detection of A549 cell cytotoxicity for submerged cultures at varying cell densities on 0.4 μm Transwell® inserts for up to 20 days. Cells were apically seeded in 200 volumes at cell densities of 2.5×10⁵ cells/cm², 1×10⁵ cells/cm² and 0.5×10⁵ cells/cm² Supernate samples (n=2) were collected from both apical (A) and basolateral (B) compartments from independent (n=4) Transwell® inserts of two cell passages. Data are expresses as mean±SD of each day's observation. Bonferroni post hoc test after two-way ANOVA where #:p<0.05, *** :p<0.0001 is shown. This demonstrates cell viability alveolar type II epithelial cells in culture over the timeframe of model construction. Increasing LDH concentrations in the apical compartment indicate apical maturation of cells as occurs in vivo. Background/low levels of LDH in basolateral compartment indicate viable cell layer adjacent to porous membrane.

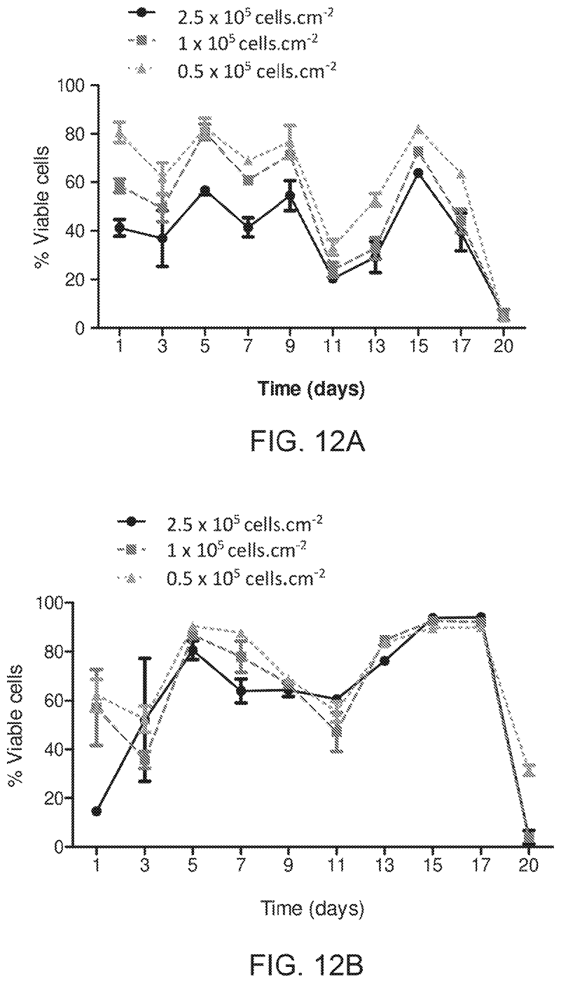

FIGS. 12A and 12B illustrate the impact of seeding density of viability profiles of alveolar epithelial cells. Seeding profiles for hAELVi (A) and A549 (B) cells grown on 96 well plates up to 20 days and cultured in hAELVi medium or U937 medium respectively. Cells were seeded in 100 μL volumes at densities of 2.5×10⁵ cell/mL, 1×10⁵ cell/mL, or 0.5×10⁵ cell/mL. Viability was assessed using flow cytometry ViaCount assay. Percentage of viable cell was determined against total cells of 1000 events. Data are represented as mean±SD for n=6. This demonstrates alveolar type I and type II cell proliferation and viability is unaffected by seeding density.

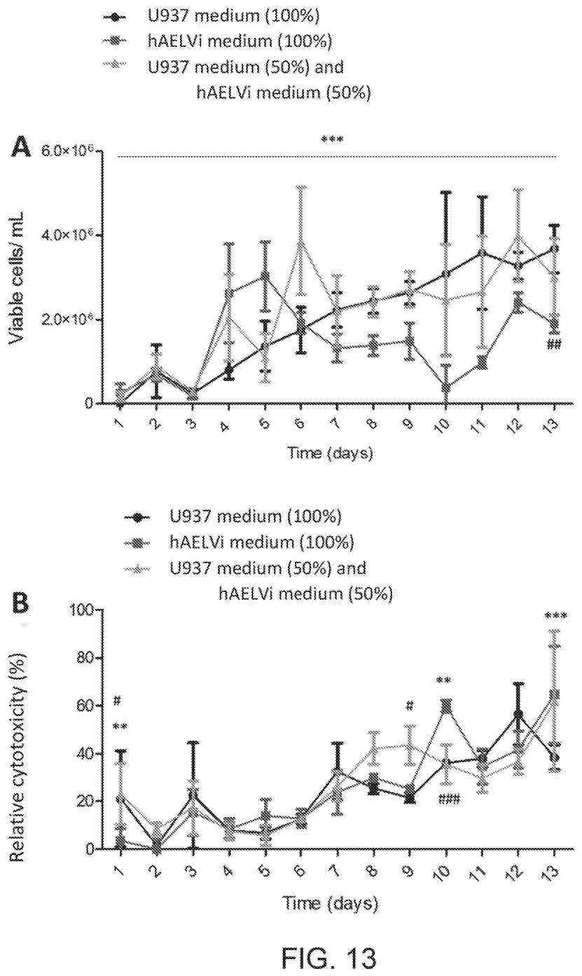

FIG. 13 illustrates the impact of different medium compositions on hAELVi cells, cultured in 96 well plate in submerged culture. Viability (A) and relative cytotoxicity (LDH release) (B) measurements of hAELVi mono-cultures incubated with U937 medium, hAELVi medium or U937 medium:hAELVi medium at a 1:1 ratio. Viable cells were measured by flow cytometry, ViaCount assay. Cytotoxicity was determined by LDH assay with positive control; 0.1% v/v Triton-X 100. Data shown represent mean±SD (n=6) from two independent experiments; *pp<0.05; **p<0.01; ***p<0.001 vs. RPMI. This demonstrates the proliferation and viability of hAELVi cells was not significantly compromised when cultured in different media.

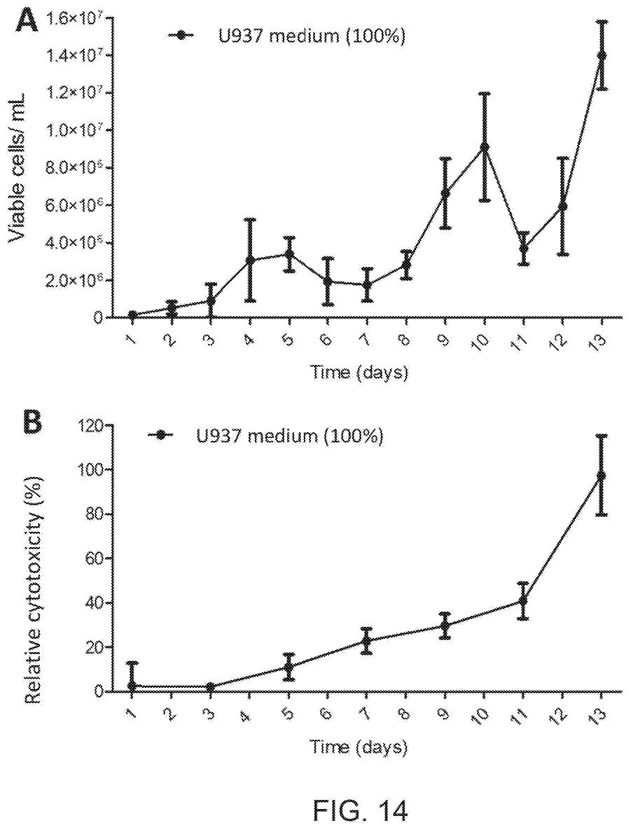

FIG. 14 illustrates the viability and relative cytotoxicity (LDH release) of A549 (B) cells cultured in different mediums for 20 days. A549 cells were cultured in U937 medium. Medium was replenished every second day. Viable cells were measured by flow cytometric, ViaCount assay. Cytotoxicity determined by LDH assay with positive control; Triton-X. Data represented as mean±SD for n=6 for 2 independent experiments. This demonstrates the proliferation and viability of A549 cells was not significantly compromised when cultured in U937 medium.

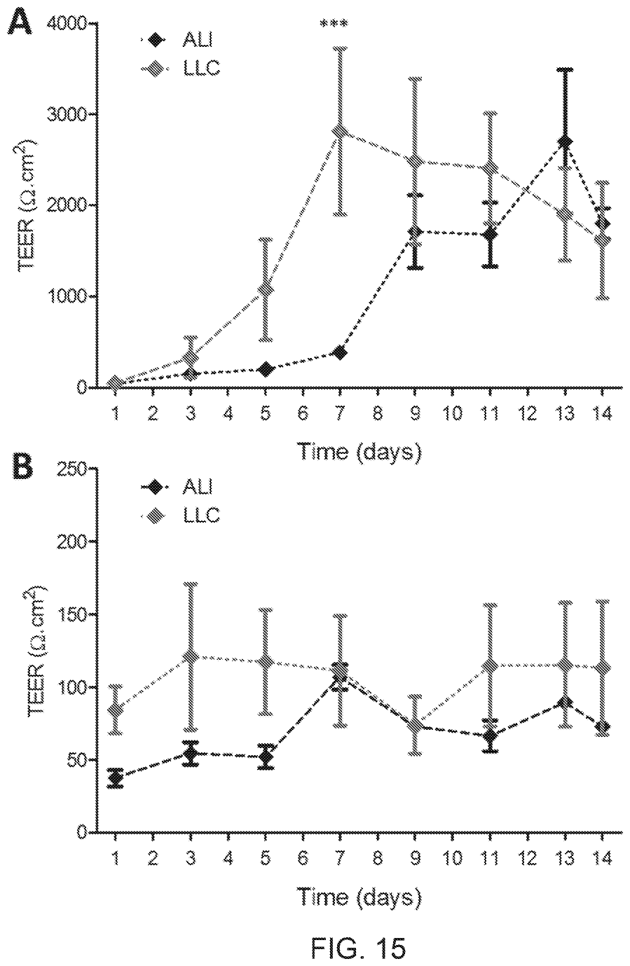

FIG. 15 illustrates epithelial cell TEER profiles cultivated at air liquid interface (ALI) and under submerged or liquid-liquid conditions (LLC). Epithelial hAELVi (A) and A549 (B) cells cultivated in hAELVi medium or U937 medium respectively. Cells were cultured under LLC and at ALI. TEER was measured every second day for 14 days. Data shown are mean±sd (n=5) independent Transwell® inserts; ***p<0.0001 vs. ALI. This demonstrates hAELVi cells (type I) cultured at ALI maintain polarised cell layers in line with in vivo alveolar epithelial cells for 9-14 days in culture. A549 cells (type II) do not form polarised cell layers as confirmed with literature.

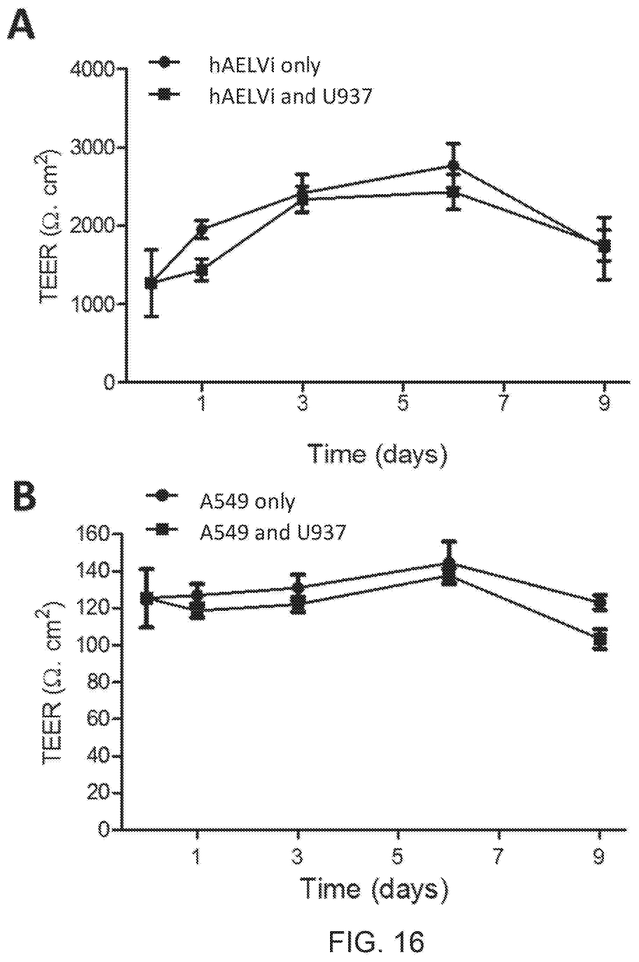

FIGS. 16-26 provide evidence to show the optimum functionality of cells in the co-cultures is maintained or improved with after model construction.

FIG. 16 illustrates the impact of macrophages on barrier function of epithelial cells. TEER measurements of (A) hAELVi (B) A549 cells in co-culture with differentiated U937 cells at ALI. Pre-construction; cells were cultivated in optimised culture conditions in either hAELVi medium or U937 medium. Cells were washed with warmed PBS. Epithelial cells on Transwell® inserts were transferred to U937 cell wells at ALI with 500 μL937 medium added in the basolateral compartments only. TEER values noted every third day for both U937 media-fed cultures Data shown represent mean±sd (n=5) of independent models.

This demonstrates that the trans epithelial electrical resistance of hAELVi cells (type I) is not significantly altered in the co-culture with the construction, change in medium or presence of U937 cells indicating they form polarised layers representative of the alveolar epithelium in vivo for at least 9 days after model construction. Whilst A549/type II cells are established not to form tight junctions, the presence of the alveolar macrophage-like cells in the co-culture did not significantly affect this feature.

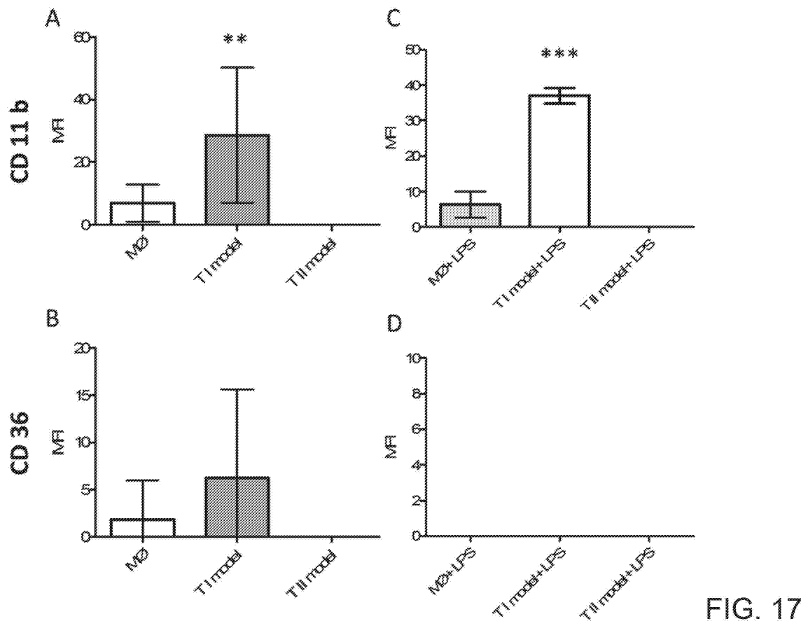

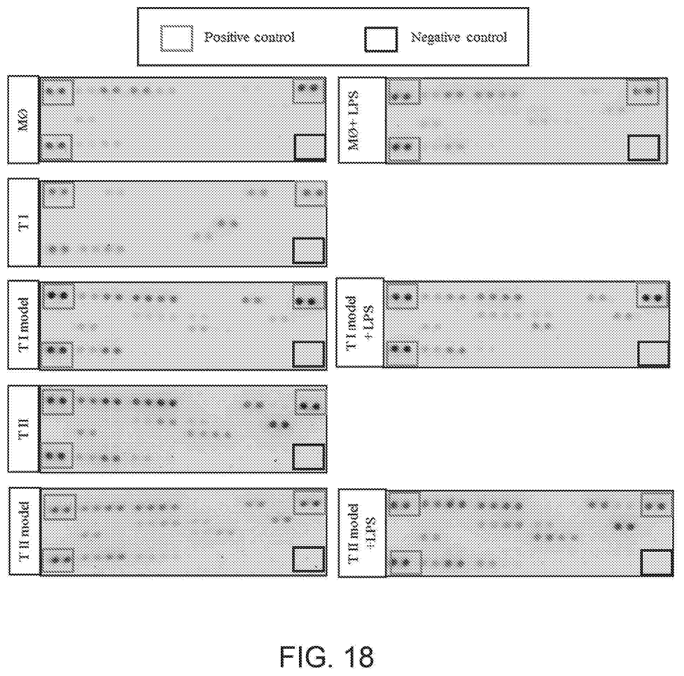

FIG. 17 illustrates CD marker expressions in LPS stimulated and non-stimulated alveoli models. Differentiated U937 cells (MØ), MØ cells cultured with hAELVi cells (T I model) or A549 cells (T II model) exposed to 10 ng/mL LPS (+LPS) for 24 h. Direct immunofluorescent staining assays for CD 11 b (A &C) and CD 36 (B & D) surface markers were performed using flow cytometry. A total of 5000 gated events were collected for each sample. MFI (median fluorescent intensity) data shown as mean±sd (n=9) for 3 independent models for Dunnett's post hoc test after one-way ANOVA where *#:p<0.005, *** :p<0.0001 vs MØ and MØ+LPS respectively. This demonstrates enhanced alveolar macrophage-like phenotypes (increase in indicative cell surface markers) when cultured with alveolar type I epithelial cells but not with alveolar type II epithelial cells. Presence of LPS is likely to drive the macrophages to M1 type activation which is in line with CD11 b expression and the absence of CD36 expression (which is an M2 marker).

FIG. 18 illustrates human cytokine profile of lower airway for ALI cell models. Detection of spots on array membranes from supernatant collect from mono-culture and co-cultures of: differentiated U937 cells (MØ) with LPS (MØ+LPS), hAELVi TI cells (T I), A549 TII cells (T II), type I co-culture model: hAELVi and MØ (T I model) with LPS (T I model+LPS), type II co-culture model: A549 and MØ (T II model) with LPS (T II model+LPS). Models were untreated or stimulated with 100 ng/mL LPS for 24 h. Controls are shown as positive (green) and negative (red). Data represents four independent Transwell® inserts. Raw data from cytokine profiling indicates that assay controls were functional and the presence of different cytokines and concentrations in different models. The data analysed in more detail in below figures.

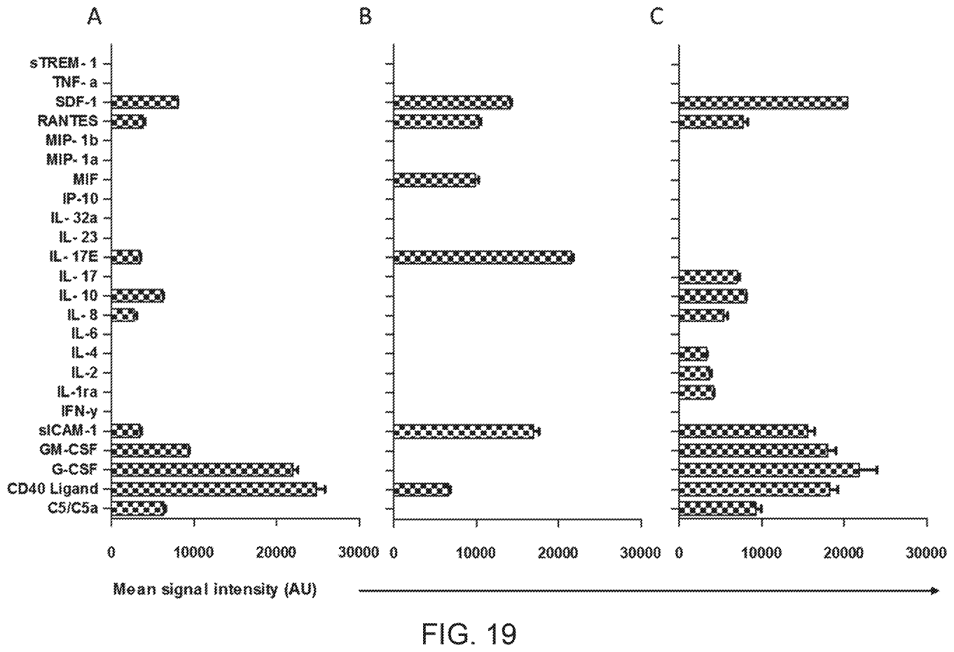

FIG. 19 illustrates human cytokine profile of lower airway T1 models. Cytokines expressed on array membranes from supernatant collected from mono-culture and co-cultures of differentiated U937 cells-MØ (A), hAELVi TI cells (B), type I co-culture model: hAELVi and MØ (C). Data represents mean signal intensity (AU) of each protein spot from the blot detected using chemiluminescence imaging and quantified using imageJ software for four independent Transwell® inserts±SD. This demonstrates quantification of cytokine profiles from FIG. 16. Co-culture model demonstrates capacity to secrete cytokines present from both mono-cultures and additional markers (e.g. IL-4, IL-2, IL-1ra, IL-17) that are not present in either model cultured alone.

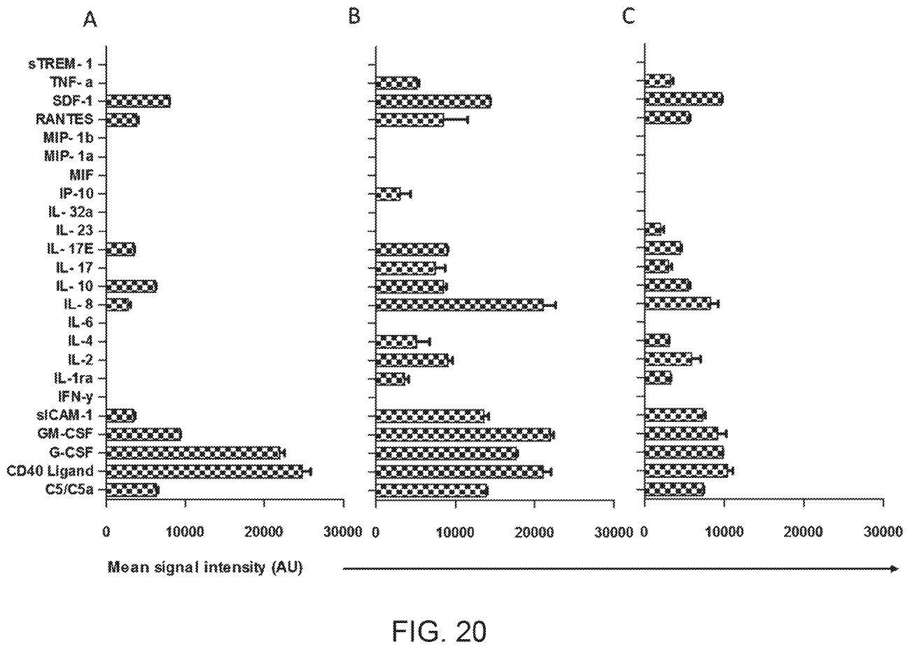

FIG. 20 illustrates human cytokine profile of lower airway T II models. Cytokines expressed on array membranes from supernatant collect from mono-culture and co-cultures of differentiated U937 cells-MØ (A), A549 T II cells (B), type II co-culture model: A549 and MØ (C). Data represents mean signal intensity (AU) of each protein spot from the blot detected using chemiluminescence imaging and quantified using imageJ software for four independent Transwell® inserts±SD. This demonstrates quantification of cytokine profiles from FIG. 16. Co-culture model demonstrates capacity to secrete cytokines present from both mono-cultures and additional markers (e.g. IL-23) that are not present in either model cultured alone.

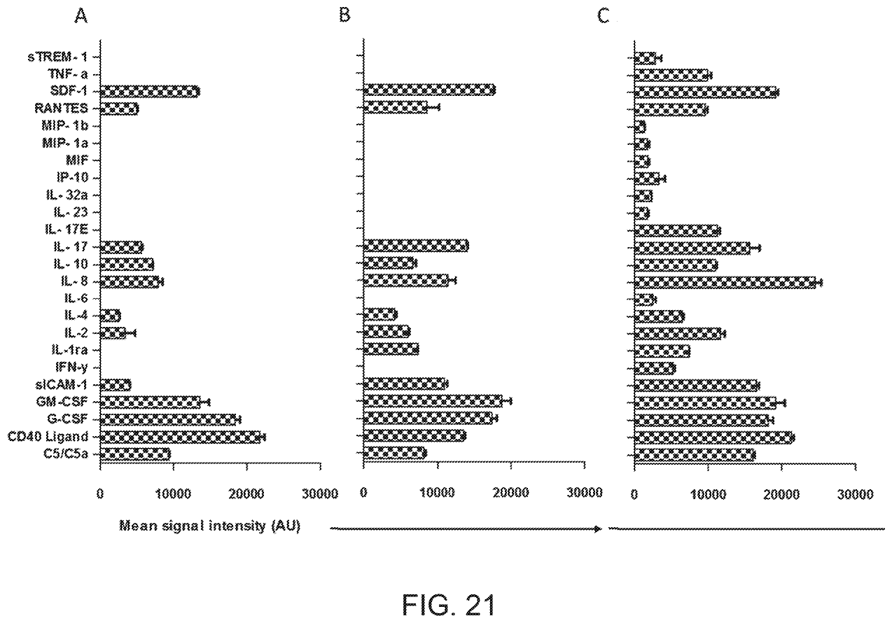

FIG. 21 illustrates human cytokine profile of lower airway; T I &T I I models in the presence of LPS. Cytokines expressed on array membranes from supernatant collect from mono-culture and co-cultures of: differentiated U937 cells (A), type II co-culture model: hAELVi and MØ (B), type I co-culture model: A549 and MØ (C) stimulated with LPS (100 ng/mL) for 24 h. Data represents mean signal intensity (AU) of each protein spot from the blot detected using chemiluminescence imaging and quantified using imageJ software for four independent Transwell® inserts±sd. This demonstrates quantification of cytokine profiles from FIG. 16. Both co-culture models demonstrate capacity to secrete cytokines present from both mono-cultures and additional markers that are not present in either model cultured alone. Markers are elevated in the presence of LPS (inflammatory stimulus).

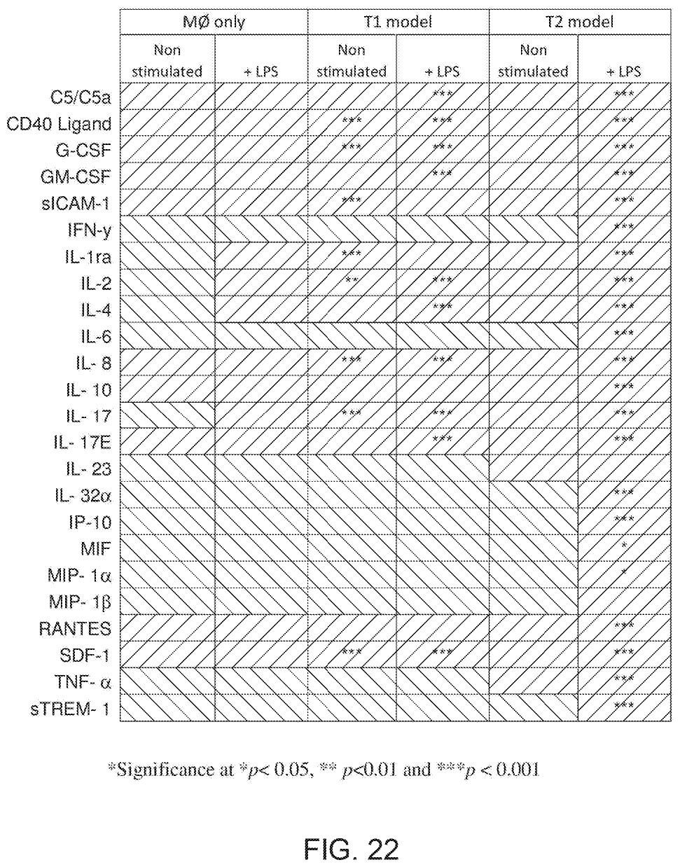

FIG. 22 illustrates human cytokine profile of lower airway of mono-culture and co-culture models in LPS. Cytokine presence in models: MØ only (differentiated U937 cells), T I model (MØ and hAELVi cells) and TII model (MØ and A549 cells) indicated in green with no detection in red. Data analysed by two-way ANOVA with Bonferroni post hoc test of non-stimulated cells vs LPS stimulation. This demonstrates significance of cytokine profiles from FIG. 19. Both co-culture models demonstrate capacity to secrete cytokines present from both mono-cultures and additional markers that are not present in either model cultured alone. Markers are elevated in the presence of LPS (inflammatory stimulus). This is particularly evident in the T2 model.

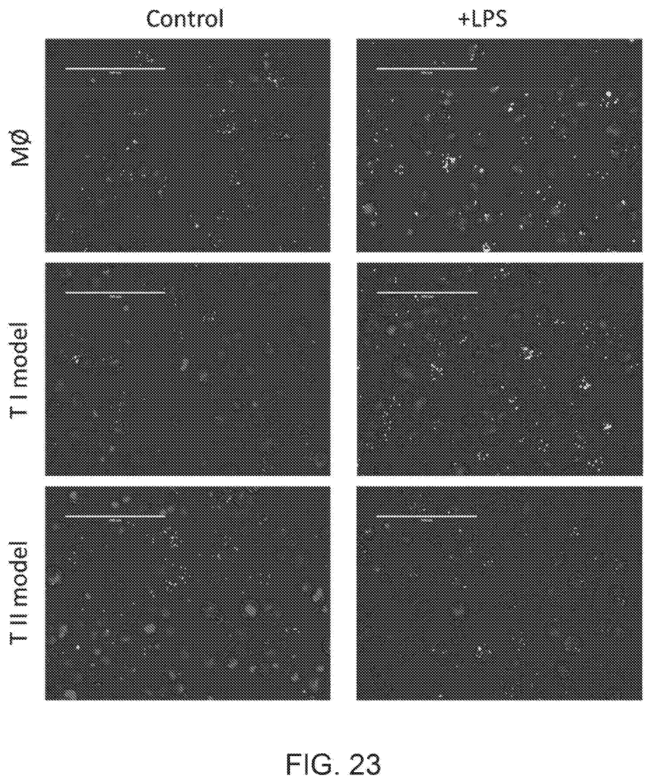

FIG. 23 illustrates a comparison of macrophages in mono-culture and co-cultures phagocytosis of microspheres. Differentiated U937 cells (MØ), MØ cells cultured with hAELVi cells (T I model) or A549 cells (T II model) exposed to 10 ng/mL LPS (+LPS) for 24 h. Phagocytosis assays were performed after 2 h incubation with 1.0 μm FluoSpheres™ Carboxylate-Modified microspheres using fluorescent microscopy. Images at 40× magnification. Scale bar is 100 μm. This demonstrates phagocytic functionality of macrophages is maintained in both co-culture models.

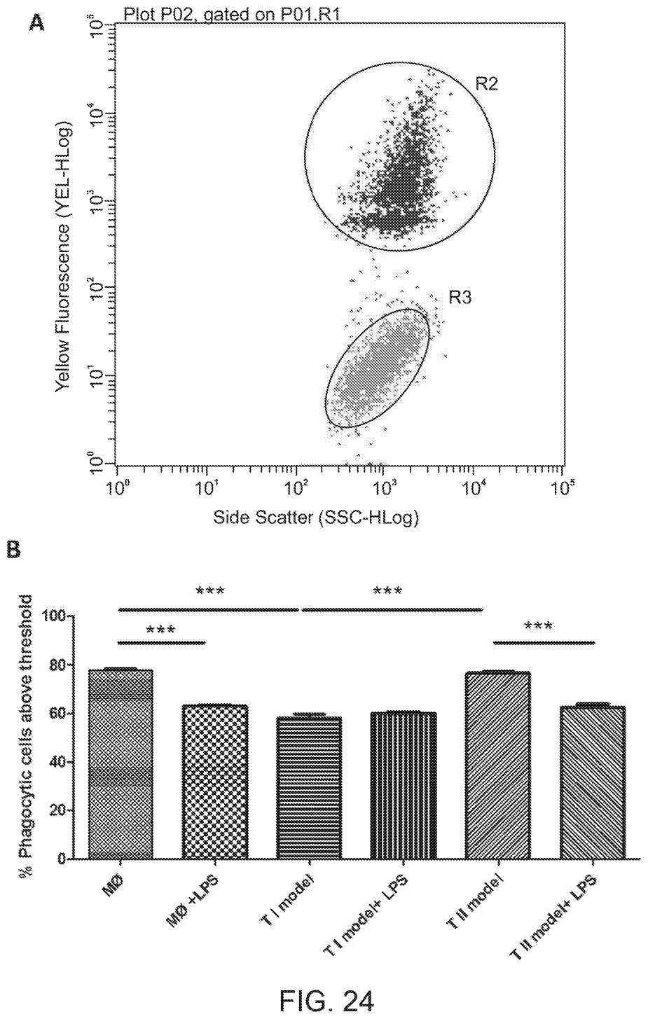

FIGS. 24A and 24B illustrate phagocytic activity of differentiated U937 cells (MØ) in mono-culture and co-cultures in the presence of LPS. MØ cells cultured with hAELVi cells (T I model) or A549 cells (T II model) exposed to 10 ng/mL LPS (+LPS) for 24 h. Phagocytosis assays were performed after 2 h incubation with 1.0 μm FluoSpheres™ Carboxylate-Modified microspheres using flow cytometry. Cells were gated for 1000 events (A) per sample. R2 shows fluorescent detection of differentiated U937 cells against side scatter above non fluorescent cells in R3. Percentage of cells above the gated (R3) threshold (B) of mono and co-cultures. A one way ANOVA was used to determine significance for each treatment group and Tukey's multiple comparison post hoc test employed when* p<0.05, *** : p<0.0001 where n=3 independent models. This demonstrates clear separation by flow cytometry of the phagocytosing and non-phagocytosing population of cells. Alveolar-like macrophage cells retain their ability to phagocytose in both co-culture systems and when challenged with LPS.

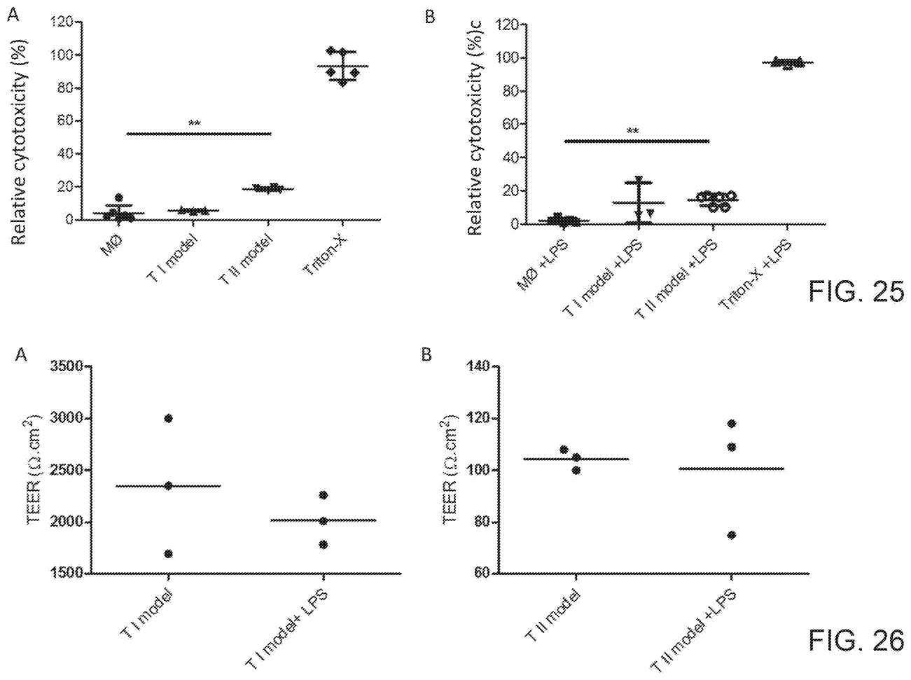

FIGS. 25A and 25B illustrate the comparison of mono-culture and co-culture construction on cell health. Percentage of maximum LDH released into supernatants from cell populations normalised to 0.1% v/v Triton-X 100 positive control. LDH released from co-cultures: (T I) hAELVi and (T II) A549 cells in co-culture with MØ cells were assessed (A). MØ cells cultured with hAELVi cells (T I model) or A549 cells (T II model) were exposed to 10 ng/mL LPS (+LPS) for 24 h (B). A one way ANOVA was used to determine significance for each treatment group and Tukey's multiple comparison post hoc test employed when* p<0.05, p<0.001 Data shown represent n=6 data points±SD of three independent cell model experiments. This demonstrates that cell viability of both cell types is unaffected by the construction of both co-culture models and is also not adversely affected in the presence of an inflammatory stimulator (LPS).

FIGS. 26A and 26B illustrate the impact of LPS on barrier properties of alveolar epithelial cells in co-culture. TEER measurements of (T I) hAELVi and (T II) A549 cells in co-culture with MØ cells. MØ cells cultured with hAELVi cells (T I model) or A549 cells (T II model) were exposed to 10 ng/mL LPS (+LPS) for 24 h. A one-way ANOVA was used to determine significance for each treatment group. Data shown represent n=3 of three independent cell model experiments (p>0.05). This demonstrates the barrier properties of the alveolar type I epithelial cells are not compromised in the co-culture set up or in the presence of LPS.

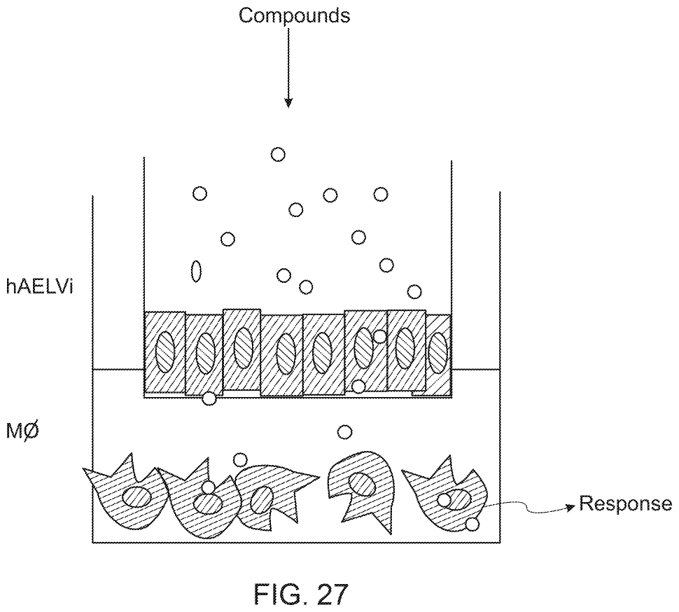

FIG. 27 illustrates a schematic representation of how the model of the present invention can be exposed to chemicals/particles for assessment.

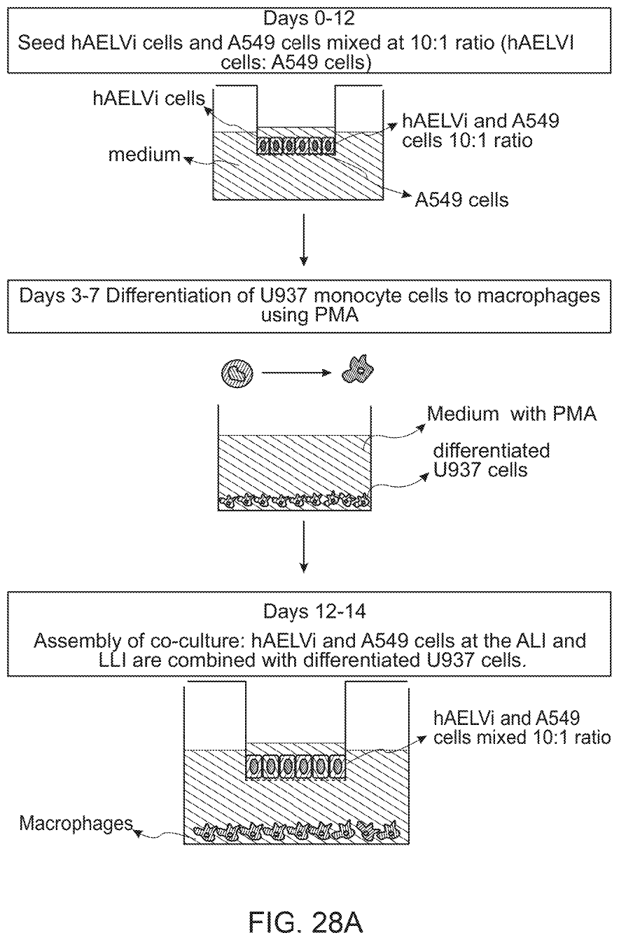

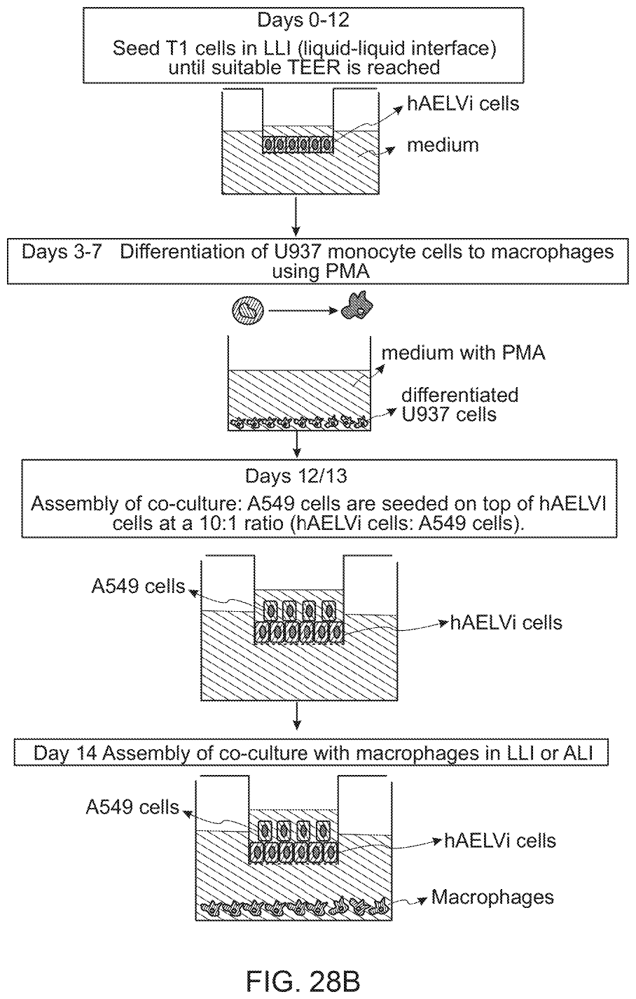

FIGS. 28-36 provide evidence to show the maintenance of cell health and epithelial barrier function improvement of response sensitivity with the combination of type 1 and type 2 epithelial cells.

FIG. 28A illustrates the generation and construct methodology for a mixed population T I and T II model which is an optimised co-culture model of mixed layer of human alveolar type I epithelial cells (hAELVi), type II epithelial cells (A549) and differentiated U937 cell (MØ). FIG. 28B illustrates the generation and construct methodology for a multi-layered population T I and T II model which is an optimised co-culture model of mixed layer of human alveolar type I epithelial cells (hAELVi), type II epithelial cells (A549) and differentiated U937 cell (MØ).

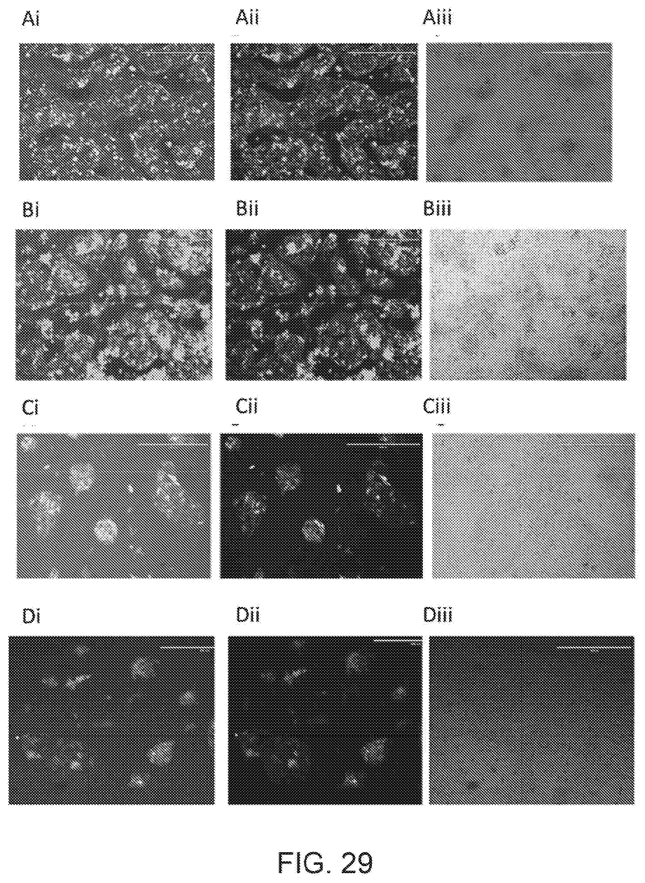

FIG. 29 illustrates the mixed population of T I and T II epithelial cells cultured at ratios of A (1:1), B (2:1), C (10:1), D (20:1) hAELVi cells:A549 cells. A549 cells were stained with CellTracker™ Green prior to seeding. (i) is an overlay image of bright field and green fluorescence (CTG/CellTracker™ Green). (ii) is the fluorescence of A549 cells with Cell Tracker™ Green, (iii) is the brightfield image.

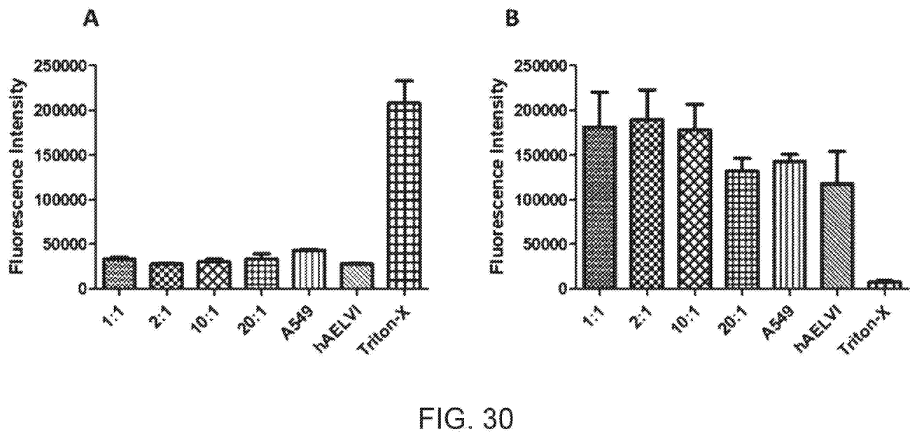

FIG. 30 illustrates viability data of hAELVi and A549 Cells cultured at different ratios in a 96 well plate. A) the viability/cytotoxicity (LDH release) of the cultured cells were assessed using LDH assay (A) and PrestoBlue™ assay (B). Mean LDH fluorescence intensity is directly proportional to cell membrane integrity. Fluorescence intensity of PrestoBlue™ is proportional to proliferation of cells. The data is represented as mean±SD. Data are presented as mean±SD, six wells were used per data point.

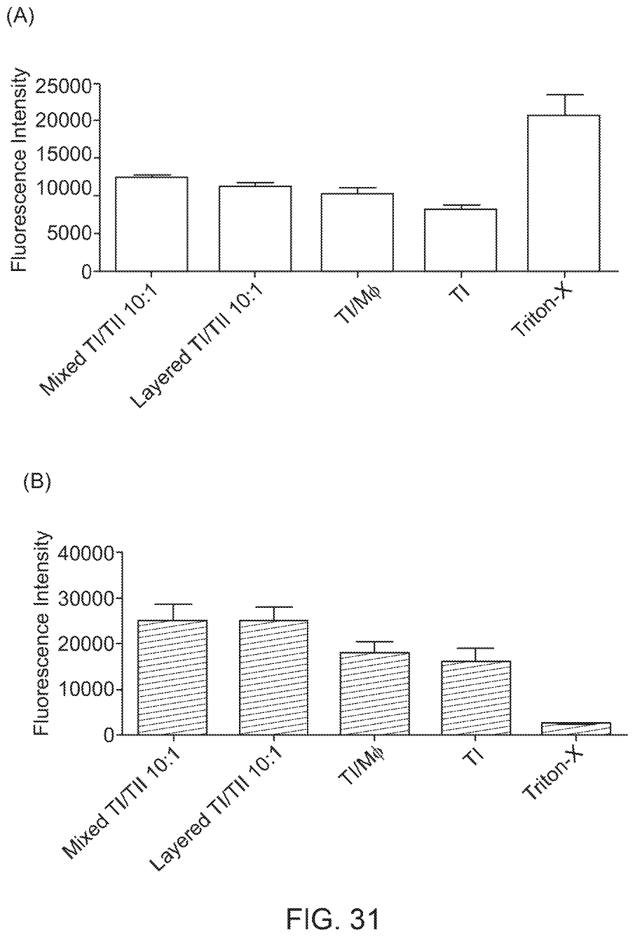

FIG. 31 illustrates viability data of hAELVi and A549 co-cultured in Transwell® inserts. A) the viability/cytotoxicity (LDH release) of the cultured cells were assessed using the LDH assay (A) and PrestoBlue™ assay (B) Mean LDH fluorescence intensity is directly proportional to cell membrane integrity. Fluorescence intensity of PrestoBlue™ is proportional to proliferation of cells. The data is represented as mean±SD. Data are presented as mean±SD, n=4.

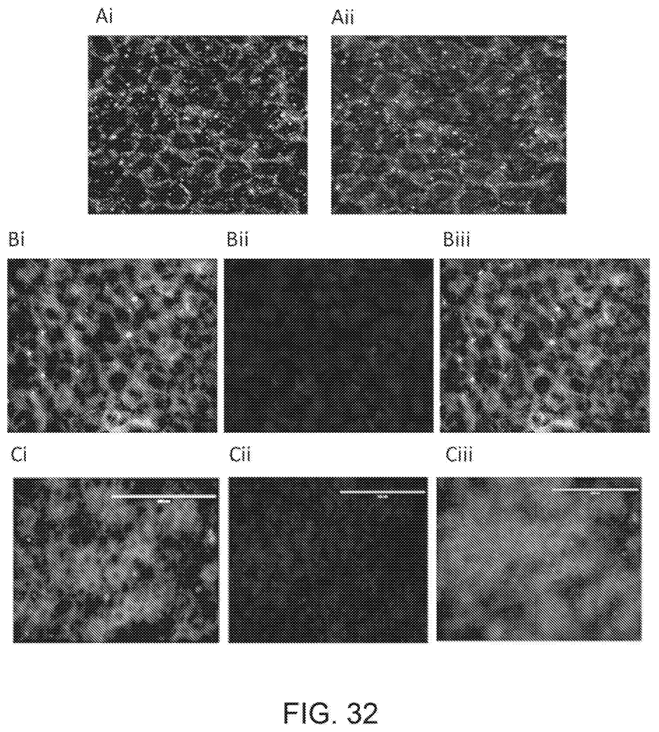

FIG. 32 illustrates the presence of tight junctions and a functional, polarised epithelial cell layer. (A) Day 14 post seeding, immunofluorescent images of hAELVi and A549 cells cultured under LLC at a 10:1 ratio in 96 well plate. (Ai) ZO-1 (Aii) Overlay of nuclear stain and ZO-1 stain. (B) Day 14 post seeding immunofluorescent images of hAELVi cells cultured under LLC. in the apical compartment of Transwell® inserts where (Bi) ZO-1 tight junction stain. (Bii) Hoechst nuclear stain (Biii) overlay of nuclear stain and tight junction stain. (C) Day 14 post seeding immunofluorescent images of A549 and hAELVi cells cultured under LLC. The hAELVi cells were previously cultured under LLC on the apical side of Transwell® inserts. A549 cells were then seeded as a layer on top using a 10:1 ratio (hAELVi:A549). (Ci) tight junction stain. (Cii) Hoechst nuclear stain (Ciii) overlay of nuclear and ZO-1 tight junction stain. Nuclear counter stain used was Hoechst 3342. Cells were labelled with anti-ZO1 tight junction primary antibody (ABCAM, ab221547) at 1/100 dilution, followed by Goat Anti-Rabbit IgG H&L (ABCAM, ab150077,Alexa Fluor® 488) secondary antibody at 1/1000 dilution (green).

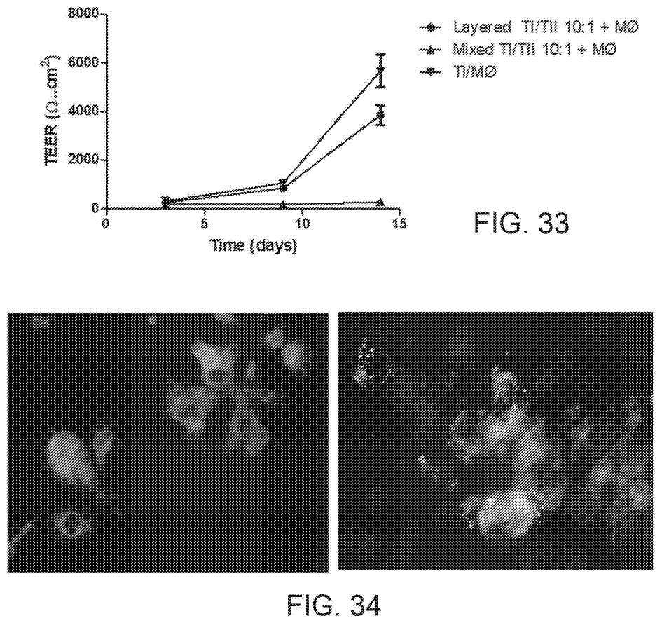

FIG. 33 illustrates the TEER values of TI/TII and differentiated U937 cells cultured in different co-culture set ups, cultured under LLC. The TEER measurements were obtained at different intervals up to 14 days. The data is presented as mean±SD, three Transwell® inserts were used per data point. The data shows that the mixed co-culture set up comprising of TI/TI (10:1) in the apical compartment and differentiated U937 cells in the bottom of the 24 well plate, had a reduced TEER value throughout. The remaining two co-culture set ups (layered TI/TII+MØ and TI/Ø) displayed the ability to form functional, polarised tight junctions.

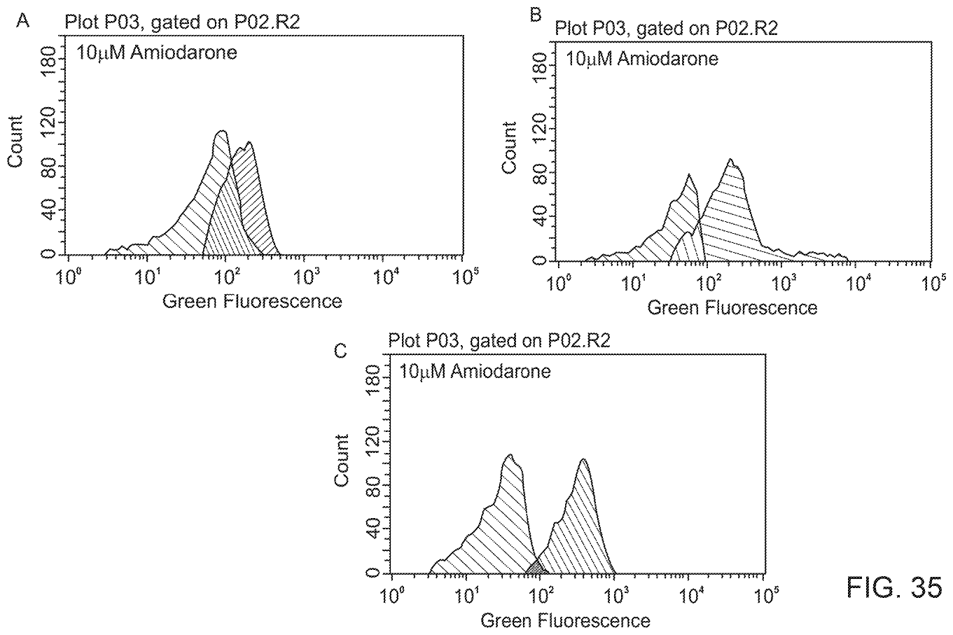

FIG. 34 illustrates surfactant protein c (SPC) production from A549 cells grown as a layer on top of hAELVi cells in ALI at a 10:1 ratio (hAELVi:A549). SPC production from A549 cells were assessed using anti-prosurfactant protein C antibody (ab90716,ABCAM) Blue—nuclei stain with Hoechst 3342, green—SPC stain).

FIG. 35 illustrates the functionality of response of alveolar macrophage-like cells to induction of phospholipidosis. Histogram represents flowcytometry data of phospholipidosis accumulation in amiodarone treated differentiated U937 cells. Differentiated U937 cells were exposed to 10 μM amiodarone. The phospholipid accumulation was assessed using HCS LipidTOX™ Green phospholipidosis detection reagent. Phospholipidosis accumulation was detected in differentiated U937 cells cultured in LLI in three different co-culture set ups: A) Type I/II cells cultured in mixed 10:1 ratio in apical compartment, differentiated U937 (lilac) cells cultured at the basolateral compartment B) Type I/II cells cultured as a layers in a 10:1 ratio in apical compartment, differentiated U937 cells cultured at the basolateral compartment (blue), C) Type I cells cultured in the apical compartment and differentiated U937 cells were seeded on the underside of Transwell® inserts (green). Pink—Untreated cells. A total of 5000 events were acquired on the flow cytometer.

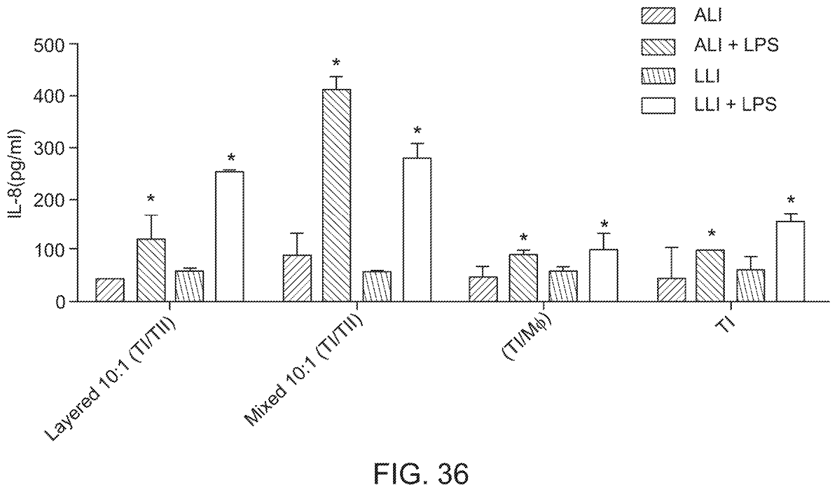

FIG. 36 illustrates extent of immune response using IL-8 secretion. IL-8 secretion was significantly increased in all models and significantly elevated in the combination type I and II epithelial models. IL-8 levels detected from supernatants of co-culture models cultured under LLI/ALI post exposure to 100 ng/ml LPS. The IL-8 levels were quantified using ELISA assay. Data shows an elevation in IL-8 levels post LPS exposure. N=4, * indicates (p<0.05).

1. A method for preparing a three-dimensional in vitro alveolar lung model comprising a culture well provided with a membrane configured to separate the culture well into a first compartment and a second compartment, wherein the membrane has first side configured form a wall of the first compartment and a second side configured to form a wall of the second compartment, wherein alveolar type I epithelial cells are provided in the first compartment and alveolar macrophage-like cells are provided in the second compartment.

2. A method as claimed in claim 1 wherein all cells are immortalized mammalian cell lines, preferably wherein all cells are immortalized human cell lines.

3. (canceled)

4. A method as claimed in claim 1 wherein the first compartment is configured to be exposed to an air-liquid interface and the second compartment configured to be submerged in a culture medium, or wherein the second compartment is configured to be exposed to an air-liquid interface and the first compartment configured to be submerged in a culture medium, or wherein both the first and second compartments are configured to be submerged in a culture medium.

5. (canceled)

6. (canceled)

7. A method as claimed in claim 1 wherein the first compartment comprises an apical compartment and the second compartment comprises a basolateral compartment, and wherein the first side of the membrane is an apical side and the second side of the membrane is a basolateral side.

8. (canceled)

9. A method as claimed in claim 1 wherein the alveolar type I epithelial cells are hAELVi cells.

10. A method as claimed in claim 1 wherein a combination of both alveolar type I epithelial cells and alveolar type II epithelial cells are provided in the first compartment, preferably wherein a combination hAELVi cells and A549 cells are provided in the first compartment.

11. (canceled)

12. A method as claimed in claim 1 comprising preparing a co-culture of a) alveolar type I epithelial cells or combination of alveolar type I and type II epithelial cells and b) alveolar macrophage-like cells.

13. A method as claimed in claim 1 comprising the following step sequence:
i) seeding the first side of the membrane with the alveolar type I epithelial cells or combination of alveolar type I and type II epithelial cells;
ii) introducing the membrane into a first culture well such that the type I epithelial cells or combination of alveolar type I and type II epithelial cells are present in the first compartment preferably at the air-liquid interface (ALI);
iii) introducing a first culture medium into the first culture well;
iv) culturing the alveolar type I epithelial cells or combination of alveolar type I and type II epithelial cells;
v) seeding a second culture well with leukocyte cells in a second culture medium;
vi) differentiating the leukocyte cells to alveolar macrophage-like cells;
vii) removing the membrane with the cultured alveolar type I epithelial cells or combination of alveolar type I and type II epithelial cells from the first culture well and introducing the membrane with the cultured alveolar type I epithelial cells or combination of alveolar type I and type II epithelial cells into the second culture medium of the second culture well such that the alveolar macrophage-like cells, present in the second compartment are preferably submerged in the second culture medium, and the alveolar type I epithelial cells or combination of alveolar type I and type II epithelial cells are present in the first compartment, preferably at the ALI.

14. A method as claimed in claim 1 any of claims 1 to 12 comprising the following step sequence:
i) seeding the first side of the membrane with the alveolar type I epithelial cells or combination of alveolar type I and type II epithelial cells;
ii) seeding the second side of the membrane with leukocyte cells;
iii) introducing a second culture medium into culture well;
iv) introducing the membrane into a culture well such that the alveolar type I epithelial cells or combination of alveolar type I and type II epithelial cells are present in the first compartment preferably at the air-liquid interface (ALI);
v) introducing a first culture medium into culture well;
vi) culturing the alveolar type I epithelial cells or combination of alveolar type I and type II epithelial cells;
vii) differentiating the leukocyte cells to alveolar macrophage-like cells.

15. A method as claimed in claim 13 or claim 14 wherein the first side of the membrane is seeded with between 1×10⁴ and 5×10⁵ alveolar type I epithelial cells or combination of alveolar type I and type II epithelial cells/cm² preferably the first side of the membrane is seeded with 1×10⁵ alveolar type I epithelial cells or combination of alveolar type I and type II epithelial cells/cm².

16. (canceled)

17. A method as claimed in claim 13 or claim 14 wherein the alveolar type I epithelial cells or combination of alveolar type I and type II epithelial cells are cultured at the air liquid interface.

18. A method as claimed in claim 13 or claim 14 wherein the culture well or second side of the membrane is seeded with 1.75×10⁵ leukocyte cells/cm².

19. A method as claimed in claim 13 or claim 14 wherein the alveolar type I epithelial cells or combination of alveolar type I and type II epithelial cells are cultured for between 4-28 days, preferably for 10 days.

20. A method as claimed in claim 13 or claim 14 wherein the method further comprises differentiating the leukocyte cells to alveolar macrophage-like cells, preferably with phorbol-12-myristate-13-acetate (PMA) or with 1, 25 dihydroxyvitamin D3, most preferably differentiated with PMA.

21. A method as claimed in claim 13 or claim 14 wherein the first culture medium comprises Dulbecco's Modified Eagle's Medium (DMEM), Dulbecco's Modified Eagle's Medium/Ham's F12 (DMEM/F12) (50:50), Roswell Park Memorial Institute-1640 (RPMI), Small Airways Growth Medium (SAGM) (Lonza), human airway epithelial cell medium (hAEC), MucilAir culture medium, SmallAir culture medium (Epithelix) or human alveolar epithelium cell culture medium (huAEC) (InSCREENeX) and more preferably RPMI or huAEC.

22. A method as claimed in claim 13 or claim 14 wherein the first culture medium comprises huAEC medium (InSCREENeX), huAEC basal supplements (bovine pituitary extract, insulin, gentamicin sulfate and amphotericin (GA-1000), retinoic acid, bovine serum albumin-fatty acid free (BSA-FAF), transferrin, triiodo-L-thyronine (T3), epinephrine, recombinant human epidermal growth factor (rhEGF)), InSCREENeX), FBS and an antibiotic/antimitotic agent, preferably the antibiotic/antimitotic agent is selected from one or more of penicillin, streptomycin, gentamicin and amphotericin.

23. A method as claimed in claim 13 or claim 14 wherein the second culture medium comprises DMEM, DMEM/F12 (50:50), RPMI, SAGM (Lonza), hAEC, MucilAir, SmallAir (Epithelix) or huAEC (InSCREENeX) and more preferably RPMI or huAEC.

24. A method as claimed in claim 13 or claim 14 wherein the second culture medium comprises RPMI, FBS, L-glutamine and an antibiotic/antimitotic agent, preferably the antibiotic/antimitotic agent is selected from one or more of penicillin, streptomycin, gentamicin and amphotericin.

25. A method as claimed claim 1 wherein the membrane comprises a porous membrane, preferably the porous membrane is configured for potential migration of the alveolar macrophage-like cells between the second and first compartments, preferably wherein the porous membrane is provided with a plurality of pores, preferably the pores are between about 0.4-10 μm in diameter, more preferably between about 0.4-8 μm in diameter, and even more preferably between about 0.4-3 μm in diameter.

26. (canceled)

27. A method as in claim 1 wherein a perfusion system is provided to allow for circulation of the first and/or second culture mediums, in one alternative the perfusion system is an external perfusion system.

28. (canceled)

29. A method as claimed in claim 1 wherein the alveolar macrophage-like cells are U937 cells differentiated with PMA (phorbol-12-myristate-13-acetate) or with 1, 25 dihydroxyvitamin D3, most preferably differentiated with PMA.

30. A method as claimed in claim 13 or claim 14 wherein differentiation is performed over several days: preferably 1-7 days and more preferably 3 days.

31. A method as claimed in claim 8 wherein step vii) takes place about 7-14 days after the seeding of the alveolar type I epithelial cells or combination of alveolar type I and type II epithelial cells and after about 24 hours of differentiation of the alveolar macrophage-like cells.

32. A three-dimensional in vitro alveolar airway model constructed according to the method of claim 1.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method of using the three-dimensional in vitro alveolar lung model of claim 32 for assessing and/or determining and/or predicting and/or inhibiting a response of a product on the alveolar barrier of lungs.

50. A method as claimed in claim 49 comprising the steps of:
a) exposing the product to be tested on the first or apical compartment of the three-dimensional model and/or the second or basolateral compartment of the three-dimensional model;
b) image analysis techniques to evaluate morphological characteristics (for example parameters including but not limited to as cell size, cell shape, vacuole characteristics, organelle characteristics);
c) assessment of barrier function of the alveolar type I epithelial cells or the combination of both alveolar type I and type II epithelial cells (for example parameters including but not limited to transepithelial electrical resistance (TEER), paracellular permeability); and
d) further biological endpoints including but not limited to genotoxicity, biochemical markers of apoptosis, proteomics, transcriptomics, metabolic activation, cell membrane integrity may also be measured.

51. (canceled)

52. A method as claimed in claim 49 comprising the steps of:
a) exposing the product to be tested on the first or apical compartment of the three-dimensional model and/or the second or basolateral compartment of the three-dimensional model;
b) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
c) assessing markers for alveolar inflammation to be measured by flow cytometry or other biological assay (including but not limited to INF-gamma, TNF-alpha, IL-12, CXCL9-11, IL-8, IL-6, GM-CSF).

53. (canceled)

54. A method as claimed in claim 49 comprising the steps of:
a) exposing the product to be tested on the first or apical compartment of the three-dimensional model of the second or third aspects of the present invention and/or the second or basolateral compartment of the three-dimensional model;
b) image analysis techniques to evaluate morphological characteristics (for example parameters including but not limited to as cell size, cell shape, vacuole characteristics, organelle characteristics);
c) assessment of barrier function of the alveolar epithelial component (for example parameters including but not limited to transepithelial electrical resistance (TEER), paracellular permeability);
d) further biological endpoints including but not limited to genotoxicity, biochemical markers of apoptosis, proteomics, transcriptomics, metabolic activation, macrophage (or cell) migration, cell membrane integrity may also be measured;
e) assessing markers for alveolar macrophage activation to be measured by flow cytometry (for example including but not limited to CXCL9, CXCL10, CXCL11, IL-12, IL-4, IL-13, IL-10, Arg1, CD206, FIZZ-1); and
f) assessing markers for alveolar inflammation to be measured by flow cytometry or other biological assay (including but not limited to INF-gamma, TNF-alpha, IL-12, CXCL9-11, IL-8, IL-6, GM-CSF).

55. (canceled)

56. A method as claimed in claim 49 wherein the response is a toxicological response, an inflammatory response, a biological response, a pharmacological response, or a biochemical response.

57. A method of using the three-dimensional in vitro alveolar lung model of claim 32 for assessing a product.

58. A method as claimed in claim 57 for assessing the fate of the product in the alveolar environment in the lungs.

59. A method as claimed in claim 57 comprising the steps of:
a) exposing the product on the first or apical compartment of the three-dimensional model;
b) assessing the concentration of the product and product metabolites within the model by an appropriate analytical tool (for example including but not limited to fluorescence, radiochemistry, LC-MS, HPLC);
c) assessing the localisation of the product and product metabolites within the model by an appropriate analytical tool (for example including but not limited to fluorescence microscopy, radiochemistry, image flow cytometry, SEM, TEM); and
d) assessing the physical characteristics (e.g. agglomeration) of the product within the model by an appropriate analytical tool (for example including but not limited to microscopy, SEM, TEM).

60. A kit of parts for creating a three-dimensional in vitro alveolar airway model according to claim 32 comprising:
alveolar type I cells or a combination of both alveolar type I and type II epithelial cells;
alveolar macrophage-like cells;
cell culture medium;
cell culture supplements;
culture vessel; and
assembly instructions.
